(19) United States
(12) Patent Application Publication
(10) Pub. No.: US 2025/0197944 A1
(43) Pub. Date: Jun. 19, 2025

(54) DIFFERENTIAL DIAGNOSIS METHOD

(71) Applicant: BELGIAN VOLITION SRL, Isnes (BE)

(72) Inventors: Theresa K. KELLY, Isnes (BE); Jacob Vincent MICALLEF, Isnes (BE); Shane G. POPLAWSKI, Isnes (BE)

(21) Appl. No.: 18/845,506

(22) PCT Filed: Mar. 10, 2023
(86) PCT No.: PCT/EP2023/056233

§ 371 Date: Sep. 10, 2024

Related U.S. Application Data
(60) Provisional application No. 63/319,067, filed on Mar. 11, 2022.

(51) Int. Cl. C12Q 1/6886; C12Q 1/6804; C12Q 1/6869

(52) U.S. Cl. CPC C12Q 1/6886; C12Q 1/6804; C12Q 1/6869; C12Q 2563/173; C12Q 2600/112

(57) ABSTRACT

The invention relates to methods and uses of the size profile of circulating chromatin fragments, nucleosomes or cfDNA present in a blood, serum or plasma sample as a biomarker for the differential diagnosis of cancer or a NETosis related disease, e.g. sepsis and a method for measuring nucleosomes in a sample using a DNA intercalating dye.

### FIELD OF THE INVENTION

The present invention relates to using the size profile of cell free nucleosomes and DNA as biomarkers for the differential detection of inflammatory and cancer diseases.

### BACKGROUND OF THE INVENTION

DNA abnormalities are characteristic of all cancer diseases. The DNA of cancer cells differs from that of healthy cells in many ways including, but not limited to, point mutations, translocations, gene copy number, micro-satellite abnormalities, DNA strand integrity and DNA methylation patterns. Tumour genetic and epigenetic characteristics vary between different tumour types and between different patients with the same tumour disease. Moreover, these characteristics vary over time within the same cancer of the same patient with the progression of the disease and in the development of acquired resistance to drug or other therapies. Thus, serial investigation of tumour DNA may help the clinician to assess minimal residual disease, predict patient prognosis, select appropriate treatments for the patient, monitor disease progression and detect any relapse or acquired treatment resistance at an early stage (possibly many months earlier than radiological detection) and allow potentially successful changes in treatment courses. DNA obtained from cancer cells or tissue removed at biopsy or surgery is investigated routinely for clinical diagnostic, prognostic and treatment selection purposes.

It is well known that some DNA that originates from cancer cells is also found in the bloodstream. Analysis of this circulating tumour DNA (ctDNA) is termed liquid biopsy, and has the advantages associated with the minimally invasive nature of blood tests.

However, the ctDNA present in the blood of cancer patients constitutes only a part of the total cell free DNA (cfDNA) present in the circulation. The majority of cfDNA circulates in the form of cell free nucleosomes (cf-nucleosomes). The majority (67.5-80%) of cfDNA is reported to circulate as mononucleosomes comprising short double-stranded DNA fragments of less than 200 base-pairs (bp) in length (see for example, Sanchez et al. JCI Insight, Am Soc Clin Invest, 2021, 6. doi.org/10.1172/jci. insight.144561 and Snyder et al. Cell. 2016; 164(1-2):57-68. doi:10.1016/j.cell.2015.11.050). The level of circulating cfDNA and cf-nucleosomes is low in healthy subjects (up to 84 ng/ml in the healthy subjects investigated here).

The level of circulating cfDNA and cf-nucleosomes is reported to be elevated in many cancers (Holdenrieder et al., Int. J. Cancer (2001) 95: 114-120). The measurement of cf-nucleosome levels, rather than cfDNA levels, has advantages of speed, cost, the low volume of plasma or serum required and the avoidance of a DNA extraction step.

Although elevated cfDNA and cf-nucleosome levels have been observed for all or most cancers, levels are not equally elevated in all cancers but vary with the type of cancer and with the stage of the disease. We have observed previously that circulating cf-nucleosome levels are highly elevated in haematological cancers (up to approximately 700 ng/ml) but are lower in solid cancers (WO2021110776).

At the early stages (stage 0 or stage 1) of solid cancer diseases, cfDNA and cf-nucleosome levels are often indistinguishable from the background levels present in healthy subjects. The proportion of cfDNA fragments in the circulation of a patient with an early stage solid cancer that is tumour derived (i.e. ctDNA) is likely to be low and the cfDNA present may comprise less than 1% ctDNA. Circulating cfDNA, ctDNA and cf-nucleosome levels tend to increase progressively with disease stages II, III, and IV. The observed levels may be several hundred ng/ml in some patients with stage IV disease, particularly in patients with a high tumour burden.

Because cfDNA and cf-nucleosome levels are often not raised in early stage solid cancer, they have not been used clinically as biomarkers to diagnose early stage cancer. However, the higher levels of cfDNA present in later stage cancer have facilitated the use of liquid biopsy methods to sequence cfDNA to characterise cancers and select optimal personalised treatment regimes.

A further factor limiting the use of cfDNA and cf-nucleosome levels as biomarkers is the non-specific nature of a finding of elevated levels in the circulation. As well as cancer, elevated levels have been observed in a wide variety of inflammatory disease conditions. Levels of circulating cfDNA and cf-nucleosomes are particularly elevated in patients with conditions involving an overreaction of the body's immune system leading to a cytokine storm in conditions such as systemic inflammatory response syndrome (SIRS), influenza, sepsis, bird-flu, pneumonia, COVID-19, acute respiratory syndrome (ARS), acute respiratory distress syndrome (ARDS), Middle-East respiratory syndrome (MERS) or severe acute respiratory syndrome (SARS) and many more. In particular, circulating cfDNA and cf-nucleosome levels have been observed in moderate sepsis disease similar to those observed in haematological cancers. Levels are similarly highly elevated in COVID-19, sepsis, SIRS, ARS, ARDS and SARS. It has been shown that cf-nucleosome levels in sepsis and COVID-19 increase with the severity of the disease and may exceed 1000 ng/ml in subjects requiring hospitalisation or exceed 2000 ng/ml in subjects requiring intensive care and organ support (Cavalier et al. Front. Mol. Biosci. (2021) 8:600881. doi: 10.3389/fmolb.2021.600881). Sepsis, SIRS, ARS, ARDS or SARS are often complications of a viral or bacterial infection, for example influenza. In these diseases the origin of the elevated cfDNA and cf-nucleosome levels does not lie in cancer cells but in extracellular traps (ETs), most commonly neutrophil extracellular traps (NETs), produced by NETosis as part of the innate inflammatory response. ETs and NETs consist of chromatin material which has been ejected into the extracellular space to trap and kill pathogens. Inflammatory diseases such as sepsis, SIRS, ARS, ARDS or SARS are characterised by an inappropriately high level of NETosis leading to release of high levels of NET material.

Therefore, a further difficulty in the use of cfDNA or cf-nucleosome levels for the detection and assessment of cancer diseases or inflammatory diseases such as sepsis lies in the non-specific nature of the finding of an elevated level and the resulting lack of differential diagnosis. Moreover, these diseases all tend to display non-specific symptoms, especially at early stages, so that, for example, non-specific cancer symptoms may be difficult to differentiate from non-specific symptoms resulting from an overreactive immune response.

This non-specific nature of a finding of elevated cfDNA or cf-nucleosome levels is a serious problem because cancer causes approximately 10 million deaths per annum worldwide. Sepsis affects some 49 million people and is the cause of approximately 11 million deaths per annum worldwide.

There remains a need in the art to provide simple, cost-effective methods to identify patients with elevated cfDNA or cf-nucleosome levels and to determine whether the elevation relates to a cancer or an inflammatory NETosis related condition. We now report methods for determining whether circulating chromatin fragments present in a blood sample have a cancer origin or an inflammatory origin from ETs or NETs.

### SUMMARY OF THE INVENTION

According to a first aspect, there is provided the use of a size profile of circulating chromatin fragments, cell free nucleosomes (cf-nucleosomes) or cell free DNA (cfDNA) present in a blood, serum or plasma sample as a biomarker for the differential diagnosis of cancer or a NETosis related disease.

According to a further aspect of the invention, there is provided a method for the differential diagnosis of cancer and a NETosis related disease in a subject which comprises the steps of:
(i) determining the size profile of circulating chromatin fragments in a blood, serum or plasma sample obtained from the subject, wherein the subject has been determined to have an elevated level of circulating chromatin fragments; and
(ii) using the size profile of the chromatin fragments to indicate whether the disease present is cancer or a NETosis related disease, wherein the size profile of the circulating chromatin fragments present in a subject with a NETosis related disease is larger compared to a subject with cancer.

According to a further aspect of the invention, there is provided a method of treating a cancer in a subject, which comprises the following steps:
(i) determining the size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is a cancer; wherein the size profile of the circulating chromatin fragments in a subject with a cancer is smaller compared to a subject with a NETosis related disease; and
(iii) administering a treatment to the subject if they are determined to have a cancer in step (ii).

According to a further aspect of the invention, there is provided a method of treating a NETosis related disease in a subject, which comprises the following steps:
(i) determining the size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is a NETosis related disease; wherein the size profile of the circulating chromatin fragments in a subject with a NETosis related disease is larger compared to a subject with cancer; and
(iii) administering a treatment to the subject if they are determined to have a NETosis related disease in step (ii).

According to a further aspect of the invention, there is provided a method for measuring nucleosomes in a sample which comprises the steps of:
(i) contacting the sample with a binding agent which specifically binds to a nucleosome or a component thereof;
(ii) contacting the nucleosomes bound in step (i) with a DNA intercalating dye;
(iii) determining the degree of binding of the DNA intercalating dye to the nucleosomes; and
(iv) using the degree of binding of the DNA intercalating dye to measure the amount of nucleosomes present in the sample.

According to a further aspect of the invention, there is provided a method for measuring nucleosomes in a sample which comprises the steps of:
(i) contacting the sample with a DNA intercalating dye;
(ii) contacting the sample with a binding agent which specifically binds to a nucleosome or a component thereof;
(iii) determining the degree of binding of the DNA intercalating dye to the nucleosomes; and
(iv) using the degree of binding of the DNA intercalating dye to measure the amount of nucleosomes present in the sample.

### BRIEF DESCRIPTION OF THE FIGURES

FIG. 1: EDTA plasma concentrations of nucleosomes containing histone H3.1 (H3.1-nucleosomes) measured in patients with a variety of cancer diseases and with moderate COVID-19 disease.

FIG. 2: Electropherograms for serum samples collected from 3 healthy subjects. Whole blood was collected into serum blood collection tubes (BCTs) and left as whole blood for 20 minutes, 24 hours or 48 hours prior to centrifugation and separation of the serum. The 3 serum samples separated at 20 minutes contain a small mononucleosome peak. The serum samples separated at 24 and 48 hours contain large chromatin fragments produced by coagulation induced NETosis, as well as large mononucleosome and oligonucleosome fragments produced as metabolites of NETs digestion.

FIG. 3: Electropherograms obtained for:
(A) 5 EDTA plasma samples obtained from solid cancer patients with moderately elevated H3.1-nucleosome levels up to 350 ng/ml. The circulating chromatin fragments present in the cancer plasma samples comprise primarily mononucleosomes and some oligonucleosomes. Peaks corresponding to large chromatin fragments are either absent or small indicating that the nucleosomes are primarily cancer associated. In contrast, all sepsis samples with similar H3.1-nucleosome levels investigated contained large components of large chromatin fragments produced by NETosis (FIG. 5).
(B) 3 EDTA plasma samples obtained from solid cancer patients with highly elevated H3.1-nucleosome levels >350 ng/ml. The circulating chromatin fragments present in the cancer plasma samples comprise primarily mononucleosomes but also some large chromatin fragments indicating that the tumour in these patients has an inflammatory component. In contrast, the large chromatin fragments are the primary component of the total circulating chromatin fragments observed in sepsis patients with similar H3.1-nucleosome levels (FIG. 6).

In each electropherogram the disease condition and H3.1-nucleosome level of the sample are provided in ng/ml. Abbreviations: GC: Gastric Cancer; BC: Breast Cancer; CRC: Colorectal Cancer; EC: Endometrial Cancer; RC: Renal cancer.

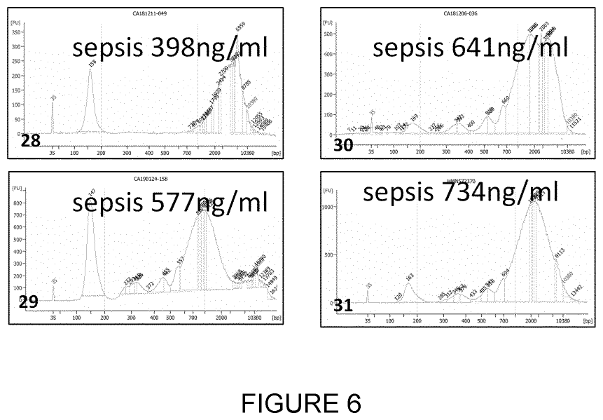

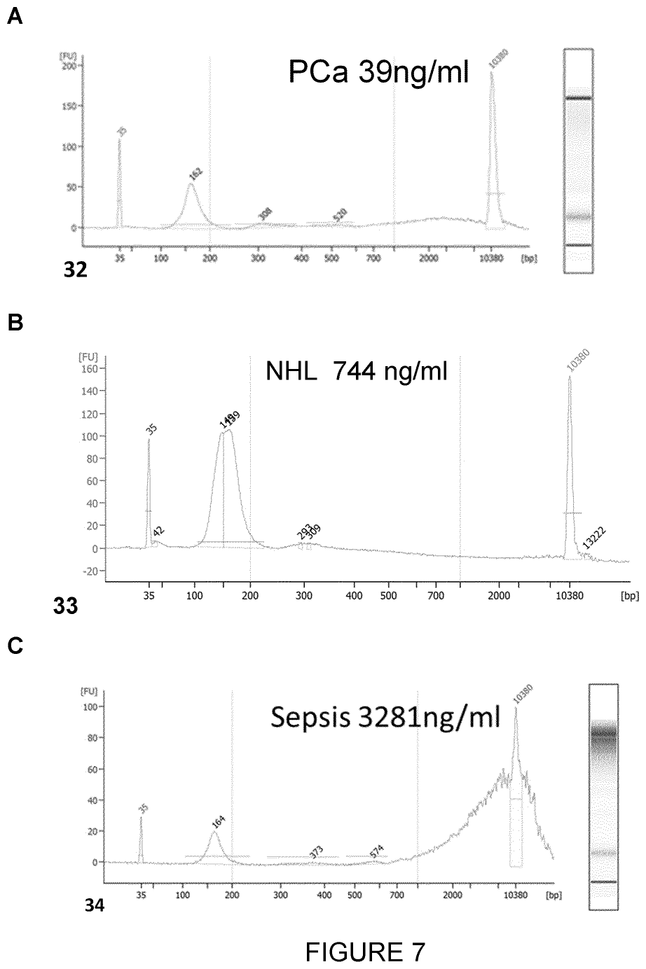

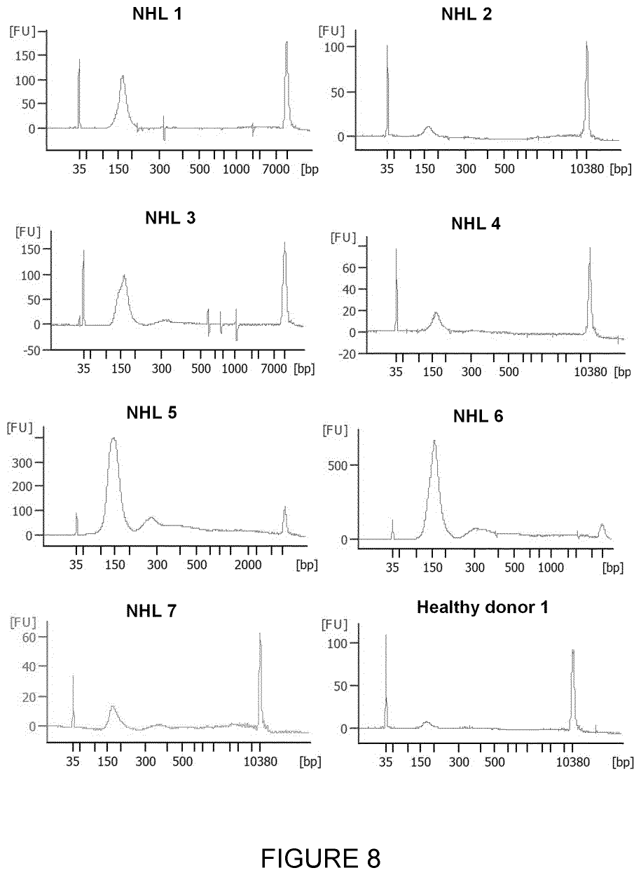

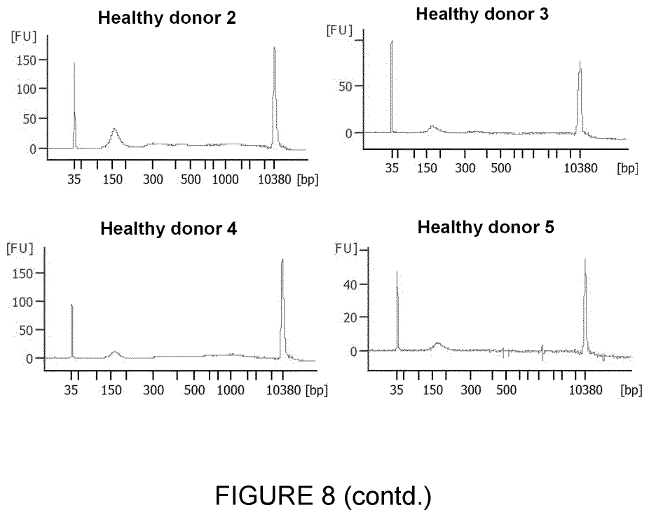

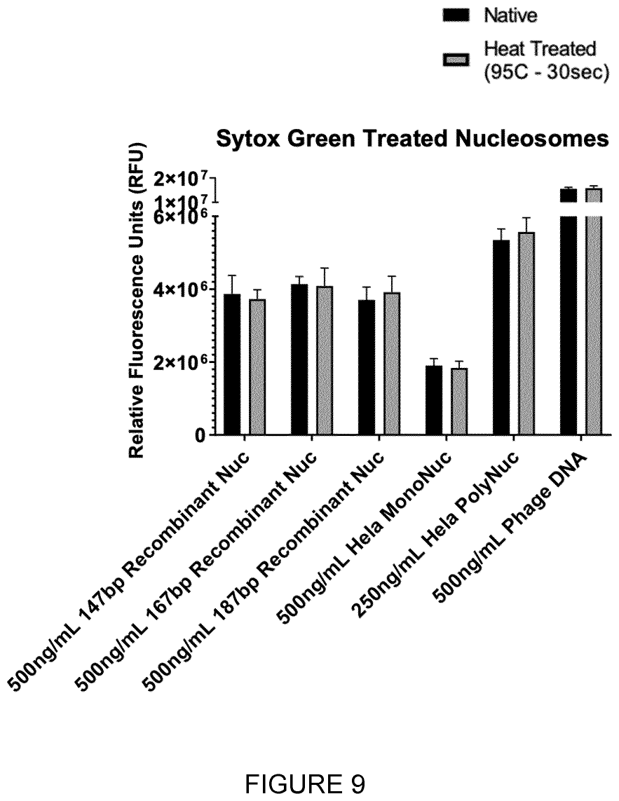

FIG. 4: Electropherograms obtained for EDTA plasma samples obtained from patients diagnosed with sepsis found to have normal or very slightly elevated H3.1 nucleosome levels.
(A) 2 EDTA plasma samples obtained from sepsis patients with low H3.1-nucleosome levels <40 ng/ml. The circulating chromatin fragments present in the sepsis plasma samples comprise a large component of, or predominantly, large chromatin fragments even at these low cf-nucleosome levels and resemble the electropherograms obtained for 24 hour or 48 hour NETosis samples in FIG. 2.
(B) 8 EDTA plasma samples obtained from sepsis patients with slightly elevated H3.1-nucleosome levels between 40-99 ng/ml. The circulating chromatin fragments present in the sepsis plasma samples comprise a large component of, or predominantly, large chromatin fragments and resemble the electropherograms obtained for 24 hour or 48 hour NETosis samples in FIG. 2.

FIG. 5: Electropherograms obtained for 9 EDTA plasma samples obtained from patients diagnosed with sepsis with moderately elevated H3.1 nucleosome levels between 100-350 ng/ml. The circulating chromatin fragments present in the sepsis plasma samples comprise a large component of, or predominantly, large chromatin fragments and resemble the electropherograms obtained for 24 hour or 48 hour NETosis samples in FIG. 2. In contrast, large chromatin fragments were absent from, or comprised a small proportion of, circulating chromatin fragments present in cancer samples with similar H3.1-nucleosome levels as shown in FIG. 3A.

FIG. 6: Electropherograms obtained for 4 EDTA plasma samples obtained from patients diagnosed with sepsis found to have highly elevated H3.1-nucleosome levels >350 ng/ml. The circulating chromatin fragments present in the sepsis plasma samples comprise a strikingly large component of large chromatin fragments and resemble the electropherograms obtained for 24 hour or 48 hour NETosis samples in FIG. 2. In contrast, cancer samples with similar H3.1-nucleosome levels comprise a small or modest component of large chromatin fragments as shown in FIG. 3B.

FIG. 7: Electropherograms obtained for 3 EDTA plasma samples obtained from patients diagnosed with (A) prostate cancer (PCa), (B) Non-Hodgkin's Lymphoma (NHL), and (C) sepsis. The circulating chromatin fragments present in the sepsis plasma sample comprise a dominant peak of large chromatin fragments above 800 bp in length indicating a NETosis origin for these fragments. In contrast, neither the PCa sample nor the NHL sample contain a significant component of large chromatin fragments despite the very high level of H3.1-nucleosomes (744 ng/ml) present in the NHL sample indicating a cancer associated origin for these chromatin fragments.

FIG. 8: Electropherograms obtained for 12 EDTA plasma samples obtained from 5 healthy donors and 7 patients diagnosed with Non-Hodgkin's Lymphoma (NHL). The circulating chromatin fragments present in all the 12 samples contained peaks corresponding only or predominantly to mononucleosomes and oligonucleosomes with no peak, or very small peaks corresponding to large chromatin fragments.

FIG. 9: Differential binding of DNA intercalating dye to mononucleosomes and polynucleosomes. The intercalating dye SYTOX™ Green was added to recombinant mononucleosomes containing 147, 167 or 187 bp of DNA, HeLa cell derived mononucleosomes or HeLa cell derived polynucleosomes. A large fluorescent signal was generated by binding of SYTOX™ Green by intercalation in 250ng of polynucleosomes. The fluorescent signal generated by binding to 500ng of recombinant mononucleosomes was lower. The signal generated by maximally incorporated dye in 500ng of phage DNA (free DNA) is also shown. The results were unaffected by a 30 second heat treatment.

### DETAILED DESCRIPTION

Cell free nucleosomes (cf-nucleosomes) are released into the circulation on cell death. In conditions involving elevated cell death, including cancer, this may lead to elevated levels of circulating cf-nucleosomes. Particularly high levels occur in haematological cancers and in late stage solid cancer.

Cf-nucleosomes are also released into the circulation in many infections that initiate cell death through a variety of mechanisms (cell binding and entry, endosomal TLR3 activation and gene expression) thereby increasing circulating cf-nucleosomes in the blood (Danthi et al., Annu. Rev. Virol. (2016) 3: 533-53).

In addition, cf-nucleosomes are also released into the circulation by the innate immune system in response to infection or other stimuli to produce extracellular traps (ETs), particularly neutrophil extracellular traps (NETs). During NETosis, intracellular chromatin in neutrophils is decondensed and released into the extracellular space, including into the circulation, as a first line response to infection. NETs and ETs comprise chromatin derived strings of cf-nucleosomes with a beads on a string structure. During NETosis the NETs are further modified by the addition of antimicrobial proteins including myeloperoxidase and neutrophil elastase which disrupt pathogens. NETs rapidly “catch” and kill pathological micro-organisms locally at the site of an infection and prevent its spread around the body.

NETs are intrinsically toxic and are metabolised by nucleases, particularly DNase, producing oligonucleosomes and mononucleosomes. Overproduction of NETs or failure to clear NETs can cause severe complications. For example, nucleosome binding to the glomerular membrane is associated with kidney damage in lupus (Kalaaji et al., Kidney Int. (2007) 71(7): 664-672), whilst NETs have been shown to intensify pulmonary injury during viral pneumonia (Ashar et al., Am. J. Pathol. (2018) 188(1): 135-148). Increasing circulating cf-nucleosome levels have been shown to correlate with increasing severity of COVID-19 disease and to be prognostic for disease severity and mortality (Cavalier et al. Front. Mol. Biosci. (2021) 8:600881. doi: 10.3389/fmolb.2021.600881). Indeed, host directed NET toxicity is associated with respiratory distress, occlusion of narrow airways, epithelial cell damage, inflammatory response and thrombus formation (Marcos et al., Nat. Med. (2010) 16: 1018-23; Hoeksema et al., Future Microbiol. (2016) 11: 441-53).

In short there are numerous sources of cf-nucleosomes in the circulation. However, these can be loosely classified into three main groups: (i) as a result of cell death during normal cell turnover, (ii) as a result of cell death during disease, and (iii) as a result of the production and metabolism of ETs by the innate immune system.

Type (i) cell death during normal cell turnover results in a background or normal level of circulating cf-nucleosomes or cf-DNA. The cellular origin of this background has been shown to be predominantly haematopoietic using deep DNA sequencing methods. In agreement with the literature (see Sanchez et al. JCI Insight, Am Soc Clin Invest, (2021) 6, and Snyder et al. Cell (2016) 164(1-2): 57-68), we have shown that this background cf-nucleosome level in healthy persons is comprised predominantly of mononucleosomes (FIG. 2).

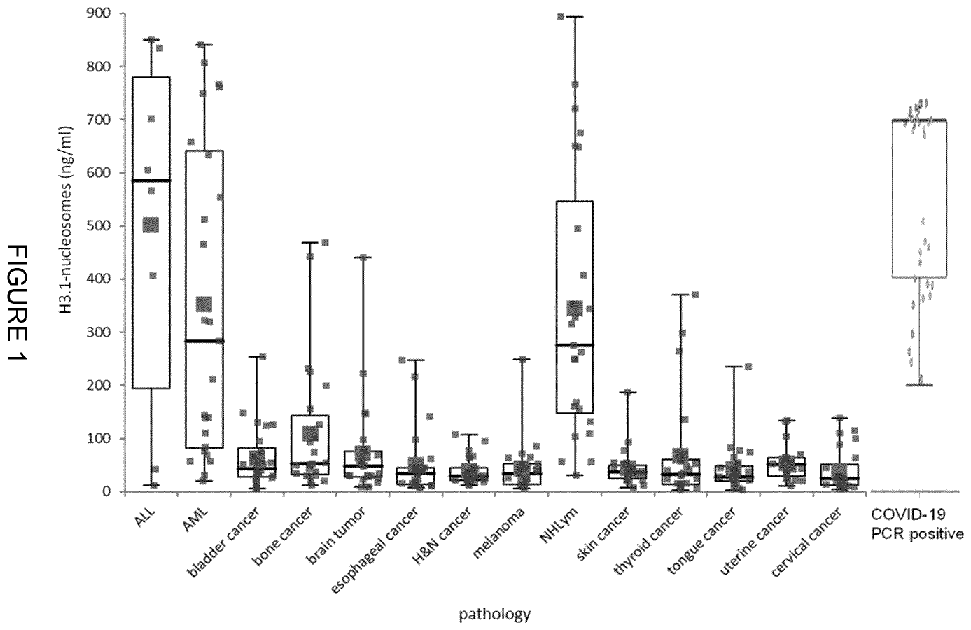

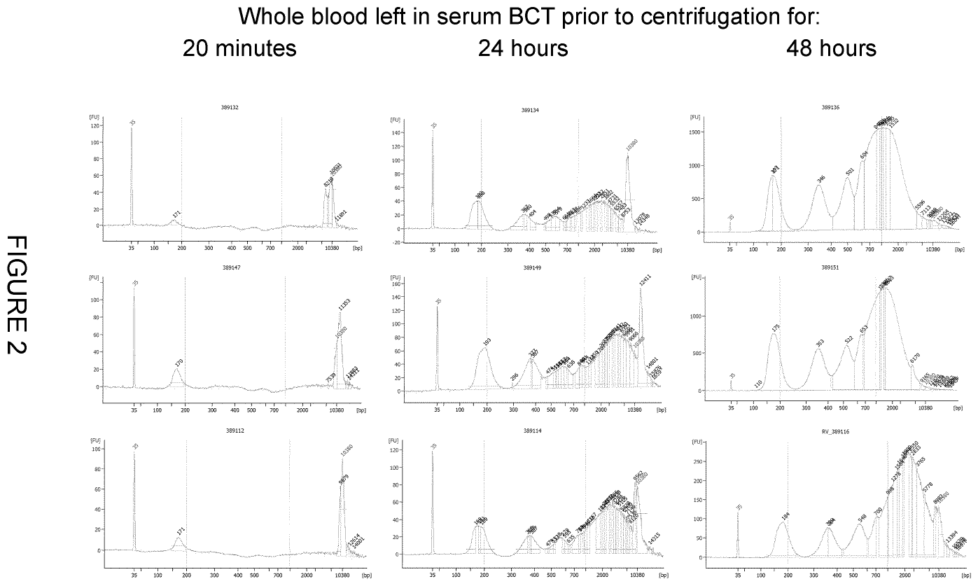

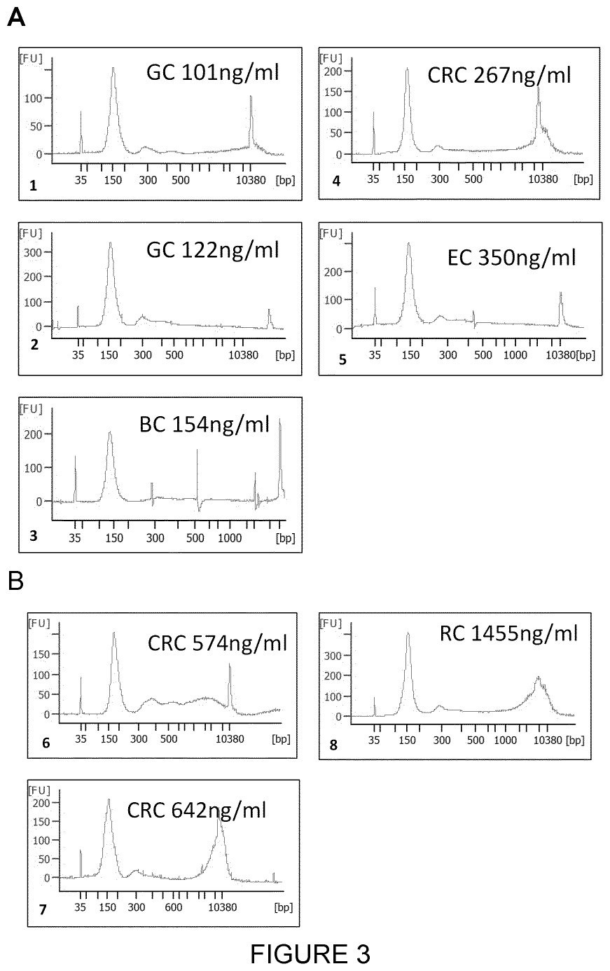

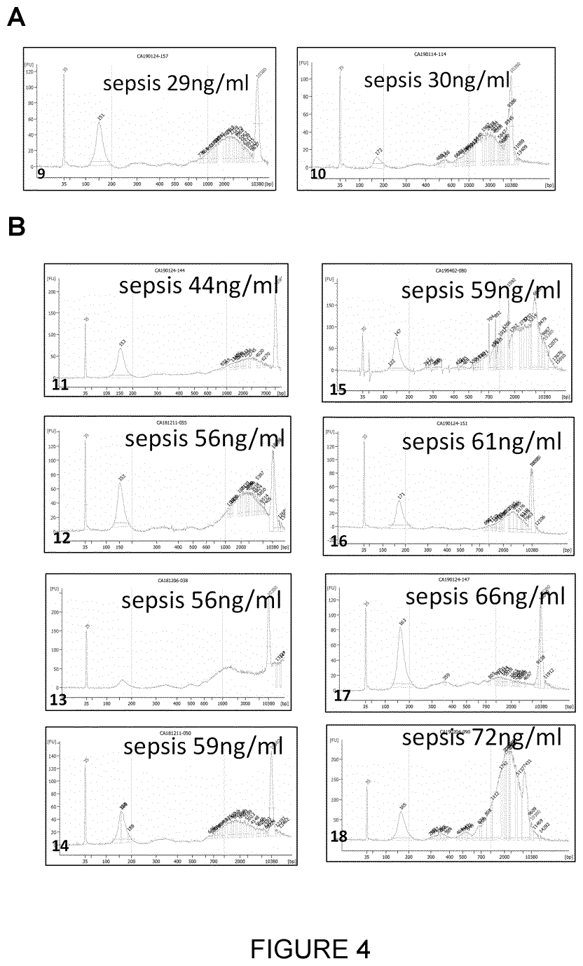

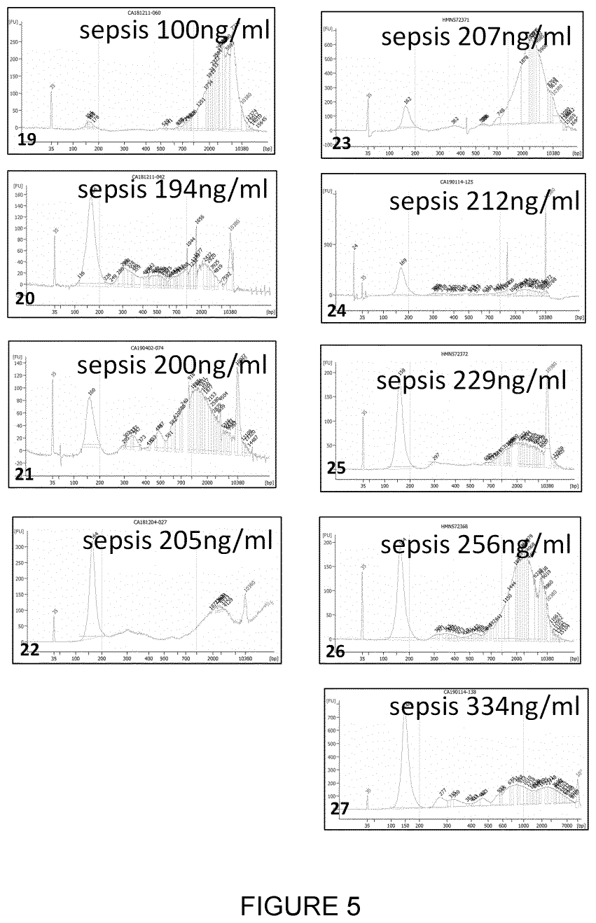

Type (ii) cell death during most diseases results in a small amount of extra cf-nucleosomes or cfDNA in the circulation. There are many disease conditions in which circulating cf-nucleosome or cfDNA levels may be slightly or moderately elevated in individuals including early stage solid cancer diseases, autoimmune disorders and many inflammatory disorders. There are also many disease conditions in which highly elevated levels of circulating cf-nucleosomes are a common finding in many or most individuals diagnosed with the disease including many late stage (solid) cancer diseases. We have also observed that haematopoietic cancers are consistently associated with highly elevated cf-nucleosome levels at all disease stages. We measured intact cf-nucleosomes (H3.1-nucleosomes) in plasma samples obtained from patients diagnosed with a variety of haematopoietic and solid cancers (FIG. 1). Similar results have been observed in dogs (Wilson-Robles et al. BMC Veterinary Research (2021) 17:231 and Dolan et al. BMC Veterinary Research (2021) 17:276). CfDNA and cf-nucleosomes can therefore be used to detect haematopoietic cancers and to monitor haematopoietic cancer diseases.

Type (iii) NETosis activity by the innate immune system results in the deposition in the circulation of large chromatin fragments consisting of long cfDNA fragments comprising many cf-nucleosomes. In conditions involving elevated NETosis, this may result in highly elevated levels of circulating cf-nucleosomes and cfDNA. We have observed that moderate COVID disease is associated with elevated cf-nucleosome levels up to 700 ng/ml or higher (FIG. 1), and that severe COVID disease involving organ failure is associated with extremely high levels of up to several thousand ng/ml (Cavalier et al. Front. Mol. Biosci. (2021) 8:600881. doi: 10.3389/fmolb.2021.600881). High levels of cf-nucleosomes and cfDNA are not only associated with severe NETosis related diseases, but are predictive of organ failure and mortality. These measurements can therefore be used to detect diseases related to NETosis such as sepsis, SIRS, COVID and many others, and to manage the treatment of such diseases.

Many questions remain surrounding the finding of an elevated cfDNA or cf-nucleosome level in a subject. It is known that at least some of the cfDNA and cf-nucleosomes present in the circulation of cancer patients carries cancer associated mutations (such as point mutations, translocations, gene copy number, micro-satellite abnormalities, DNA strand integrity or DNA methylation patterns) and has a cancer cell origin. However, it is also known that the aetiology of cancer has an inflammatory component and that the tumour environment of cancer diseases is inflammatory and can be highly inflammatory. Studies have shown a strong relationship between chronic infection, inflammation and cancer. The inflammatory tumour microenvironment and inflammatory cells and pathways are involved in the development, and metastasis of cancer. (Tan et al. Front. Pharmacol. (2021) 12: 688625 doi.org/10.3389/fphar.2021.688625). Moreover, NETs and NETosis are mechanistically involved in the establishment and progression of cancer diseases (Teijeira et al, Immunity 52, 856-871, 2020; doi.org/10.1016/j.immuni.2020.03.001). Therefore, cf-nucleosomes or cfDNA present in a sample taken from a cancer patient may conceivably include nucleosomes of either or both of a NETosis origin or cancer cell origin.

Where an elevated cfDNA or cf-nucleosome level is found in a subject diagnosed with cancer (for example a metastatic stage IV cancer), it is not known whether the cf-nucleosomes or cfDNA present are predominantly of cancer or NETosis origin or a mix of origins. Similarly, haematological cancers are known to have an inflammatory component, so it is not clear whether the cf-nucleosomes or cfDNA present in a subject with a haematological cancer are predominantly of cancer or NETosis origin or a mix of origins. We now report a method for the identification of the origin of circulating cfDNA or cf-nucleosomes in a sample as from cancer, inflammation or both.

We investigated the nature of cf-nucleosomes produced by NETosis in whole blood. We observed that coagulation of whole blood left in serum blood collection tubes (BCTs) triggers ex vivo NETosis in the tube and used this as an experimental system in which to investigate the nature, and particularly the chromatin fragment size profile, of circulating NETs. We collected whole blood samples from healthy subjects into 3 serum BCTs. We centrifuged one serum BCT containing whole blood after 30 minutes, removed the serum from the clot and analysed the serum for the size profile of cf-nucleosomes present using an electrophoresis method (the Bioanalyzer system available from Agilent Technologies). The second and third tubes were centrifuged after 24 and 48 hours respectively to allow for a large amount of NETosis and metabolism of NETs to occur and the serum was similarly analysed for chromatin fragment size profiles generated.

The resulting electropherograms (FIG. 2) show that the samples centrifuged at 30 minutes contained predominantly mononucleosomes. As NETosis is generally considered to require several hours, the mononucleosomes are representative of the cf-nucleosomes present in the circulation of the healthy donors, i.e. the background level of cf-nucleosomes. The results for EDTA plasma samples taken from healthy subjects (in which no NETosis is triggered) show a similar profile. The small nature of the fragments present in the circulation of healthy subjects may relate to the release of cf-mononucleosomes and cf-oligonucleosomes by the death of healthy cells or may relate to metabolised NETs due to digestion by nucleases. It is possible that both elements may contribute to a mixture of origins.

In contrast, the tubes centrifuged at 24 and 48 hours contained large quantities of large chromatin fragments comprising up to 10,000 bp as well as greatly increased peaks relating to mononucleosomes (approx. 180 bp), di-nucleosomes (approx. 360 bp) and tri-nucleosomes (approx. 550 bp). The presence of large chromatin fragments in the samples in which NETosis was triggered and allowed to proceed for 24 or 48 hours is indicative of a NETosis origin for these nucleosomes. It is clear that the circulating nucleases in the healthy subjects have metabolised a significant portion, but not all, of the large chromatin fragments into cf-mononucleosomes and cf-oligonucleosomes and this contributes to a characteristic NETosis cf-nucleosome/cfDNA chromatin fragment size profile. NETosis profiles generated by this or other methods may be used as reference profiles for the purposes of the invention.

We have shown that NETosis leads to the deposition of large chromatin fragments as well as mononucleosomes in the circulation (FIG. 2). We therefore hypothesised that a raised cfDNA or cf-nucleosome level observed in a blood, plasma or serum sample taken from a subject may be used to differentially indicate the presence of cancer or a NETosis related condition (for example, sepsis, thrombosis, SIRS or COVID-19 or others) on the basis of the circulating chromatin fragment size distribution present if such profiles are significantly different.

To test this hypothesis, we selected sepsis as a representative NETosis related condition and investigated EDTA plasma samples obtained from 23 patients diagnosed with sepsis. EDTA plasma was selected as the most suitable sample matrix for this purpose as the lack of a NETosis trigger means that the cf-nucleosomes observed in the samples are representative of the circulating cf-nucleosomes with no ex vivo contamination.

We measured the cf-nucleosome levels present in the sepsis samples by immunoassay and also analysed the chromatin fragment size distribution profile by electrophoresis. The electrophoresis results (electropherograms) obtained for the sepsis patients all contained a mononucleosome peak as well as a significant, wide peak corresponding to larger chromatin fragments comprising more than approximately 700 bp or 1000 bp which was usually larger in area than the mononucleosome peak present. Moreover, the electropherograms obtained for sepsis samples were similar in profile to those we obtained by ex vivo induction of NETosis in whole blood. We reasoned that the presence of such a significant, wide peak corresponding to larger chromatin fragments in a plasma sample taken from a subject was an indicator of the presence of extracellular traps (particularly neutrophil extracellular traps) and their metabolites in the circulation of the subject and an indicator of an inflammatory or NETosis process in the subject.

We then investigated EDTA plasma samples obtained from 8 patients diagnosed with a variety of solid cancers. We measured the cf-nucleosome levels present in the cancer samples by immunoassay and also analysed the chromatin fragment size distribution profile by electrophoresis. We observed that some cancer samples (5 of 8) contained primarily or only mono-nucleosome peaks but others (3 of 8) contained both mono-nucleosomes and large chromatin.

In short, we observed that cfDNA size profiles generated for samples obtained from both cancer patients and from NETosis related disease conditions may comprise both mono-nucleosomes and/or large chromatin in a sample.

We measured the H3.1-nucleosome levels of the 8 plasma samples obtained from patients diagnosed with a solid cancer disease and observed a wide range of concentrations from 101 to 1455 ng/ml. We ranked the electropherograms for the 8 plasma cancer samples by measured H3.1-nucleosome level and scrutinized the result for any systematic change in the fragment size profiles observed with H3.1-nucleosome level. Surprisingly, the nature of the chromatin fragment profile observed correlated with the cf-nucleosome levels observed. We found that electropherograms for cancer samples containing moderately elevated levels (above normal) of cf-nucleosomes up to approximately 350 ng/ml, contained a large peak corresponding to mononucleosomes and small peaks corresponding to oligonucleosomes. Peaks representing large chromatin fragments were either absent or very small indicating that the nucleosomes present in the samples were likely cancer associated and unlikely to have originated from NETosis (FIG. 3A). Electropherograms for cancer samples containing higher levels of cf-nucleosomes (>350 ng/ml) also contained a large peak corresponding to mononucleosomes but additionally contained peaks corresponding to large chromatin fragments indicating some level of inflammation and NETosis in these patients and that the cancer disease present in these patients may have had a significant inflammatory component (FIG. 3B).

We next investigated circulating chromatin fragment size profiles in EDTA plasma samples obtained from 3 patients diagnosed with sepsis, a haematological (liquid) cancer (Non-Hodgkin's lymphoma, NHL) and metastatic prostate (solid) cancer (PCa) for comparison of the profiles of solid and liquid cancers and NETosis related diseases.

The electrophoresis results for the 3 samples (FIG. 7) showed that the circulating chromatin fragments present in the sepsis plasma sample comprised a dominant peak of large chromatin fragments indicating a NETosis origin for those fragments. In contrast, the PCa sample and the NHL sample contained a strong mononucleosome peak (at approximately 160 bp) and a smaller dinucleosome peak (at approximately 300 bp) but neither contained a significant component of large chromatin fragments indicating a cancer associated origin for the chromatin fragments in these samples. The NHL sample in particular contained an extremely low proportion of large chromatin fragments despite the high level of H3.1-nucleosomes present in the sample (744 ng/ml). This is different to the pattern observed for solid cancers and is surprising given the reported inflammatory aetiology of this disease. We repeated this experiment for EDTA plasma samples obtained from several further NHL cases with highly elevated cf-nucleosome levels and confirmed the finding of a strong mononucleosome peak combined with the absence of a large chromatin peak (or the presence of only a small peak relating to large chromatin) is characteristic of liquid cancers. This illustrates the strong sensitivity and particular utility of the current invention for the differential diagnosis of a subject with an elevated level of circulating chromatin fragments that may be suspected to have an inflammatory or a haematological cancer disease.

We conclude that: (i) the finding of a chromatin fragment size distribution profile, for a plasma sample taken from a subject, that includes a significant, wide peak corresponding to larger chromatin fragments comprising more than approximately 800 bp or 1000 bp, is an indicator of an inflammatory or NETosis related process in the subject. This process may relate to a NETosis associated condition or may relate to a cancer associated inflammation. Also, (ii) the finding of an elevated chromatin fragment concentration in a plasma sample taken from a subject, in combination with a size distribution profile that includes a significant peak below 200 bp corresponding to mononucleosomes in the absence of any significant wide peak corresponding to larger chromatin fragments comprising more than approximately 800 bp or 1000 bp, is an indicator of a cancer disease in the subject. Such profiles, as exemplified using the Agilent Bioanalyzer, are shown in FIGS. 3A, 7A and 7B.

Therefore, according to a first aspect, there is provided the use of a size profile of circulating chromatin fragments, cf-nucleosomes or cfDNA present in a blood, serum or plasma sample as a biomarker for the differential diagnosis of cancer or a NETosis related disease.

In one embodiment a cancer disease profile is identified by comparison to a (reference) profile generated for a known cancer sample (for example as shown in FIGS. 3A, 7A and 7B) and/or to a (reference) profile generated for patient with a known NETosis associated disease (for example as shown in FIGS. 4, 5 and 6) and/or to a (reference) NETosis profile generated ex vivo using the same profiling method in whole blood (for example as shown in FIG. 2 for electropherograms generated by the Agilent Bioanalyzer instrument).

The results also indicate conclusion (iii), the finding of a normal or moderately elevated chromatin fragment concentration (for example up to approximately 350 ng/ml H3.1-nucleosomes) in a plasma sample taken from a subject, in combination with a fragment size distribution profile that includes both a significant peak below 200 bp corresponding to mononucleosomes, as well as a significant wide peak corresponding to larger chromatin fragments comprising more than approximately 800 bp or 1000 bp, is an indicator of a NETosis related disease in the subject. Such profiles, as exemplified using the Agilent Bioanalyzer, are shown in FIGS. 4 and 5.

Therefore, in one embodiment a NETosis related disease profile is identified by comparison to a (reference) profile generated for a known NETosis related or inflammatory condition sample (for example as shown in FIGS. 4 and 5) and/or to a (reference) profile generated for a patient with a known cancer disease with a normal or moderately elevated chromatin fragment concentration (for example as shown in FIG. 3A) and/or to a (reference) NETosis profile generated ex vivo using the same profiling method in whole blood (for example as shown in FIG. 2 for electropherograms generated by the Agilent Bioanalyzer instrument).

The results also indicate conclusion (iv), the finding of a highly elevated chromatin fragment concentration (for example greater than 350 ng/ml H3.1-nucleosomes) in a plasma sample taken from a subject, in combination with a fragment size distribution profile that includes both a significant peak below 200 bp corresponding to mononucleosomes, as well as a significant wide peak corresponding to larger chromatin fragments comprising more than approximately 800 bp or 1000 bp, wherein the wide peak corresponding to larger chromatin fragments dominates the mononucleosome peak (for example as shown in FIG. 6), is an indicator of a NETosis related disease in the subject.

Therefore, according to a further aspect, there is provided the use of the level of circulating chromatin fragments present in a blood, serum or plasma sample combined with a size profile of circulating chromatin fragments, cf-nucleosomes or cfDNA as a combined biomarker for the differential diagnosis of cancer or a NETosis related disease.

In one embodiment a NETosis related disease profile is identified by comparison to a similar profile generated for a known (or reference) NETosis related or inflammatory condition sample with a highly elevated chromatin fragment concentration (for example as shown in FIG. 6), and/or to a (reference) profile generated for a patient with a known cancer disease with a highly elevated chromatin fragment concentration (for example as shown in FIG. 3B), and/or to a (reference) NETosis profile generated ex vivo using the same profiling method in whole blood (for example as shown in FIG. 2 for electropherograms generated by the Agilent Bioanalyzer instrument).

The results also indicate conclusion (v), the finding of a highly elevated chromatin fragment concentration (for example greater 350 ng/ml H3.1-nucleosomes) in a plasma sample taken from a subject, in combination with a fragment size distribution profile that includes both a significant peak below 200 bp corresponding to mononucleosomes, as well as a significant wide peak corresponding to larger chromatin fragments comprising more than approximately 800 bp or 1000 bp, wherein the wide peak corresponding to larger chromatin fragments does not dominate the mononucleosome peak (for example as shown in FIG. 3B), is an indicator of a cancer disease in the subject. In the case of electropherograms generated using the Agilent Bioanalyzer, such mononucleosome peak may be the largest peak, or the peaks may be of comparable size (FIG. 3B).

The results further indicate that larger chromatin fragments comprising more than approximately 800 bp or 1000 bp have a high probability of a NETosis origin (i.e. they derive from NETosis or other extracellular trap material). In contrast, the results indicate that chromatin fragments comprising smaller DNA fragments of 200 bp or less corresponding to mononucleosomes have a higher probability of a non-NETosis origin.

This confirms the utility of the level of circulating chromatin fragments present in a blood, serum or plasma sample combined with a size profile of circulating chromatin fragments, cf-nucleosomes or cfDNA as a combined biomarker for the differential diagnosis of cancer or a NETosis related disease.

In one embodiment a cancer profile is identified by comparison to a (reference) profile generated for a known cancer sample with a highly elevated chromatin fragment concentration (for example as shown in FIG. 3B) and/or to a (reference) profile generated for a patient with a known NETosis related or inflammatory condition with a highly elevated chromatin fragment concentration (for example as shown in FIG. 6) and/or to a (reference) NETosis profile generated ex vivo using the same method in whole blood (for example as shown in FIG. 2 for electropherograms generated by the Agilent Bioanalyzer instrument), The results also indicate conclusion (vi), the finding of a highly elevated chromatin fragment concentration (for example than 350 ng/ml H3.1-nucleosomes) in a plasma sample taken from a subject, in combination with a size distribution profile that includes a significant peak below 200 bp corresponding to mononucleosomes in the absence of any significant wide peak corresponding to larger chromatin fragments comprising more than approximately 800 bp or 1000 bp, is an indicator of a cancer disease in the subject and that the cancer disease is a haematological cancer (for example as shown in FIG. 7B).

Therefore, according to a further aspect, there is provided the use of the level of circulating chromatin fragments present in a blood, serum or plasma sample combined with a size profile of circulating chromatin fragments, cf-nucleosomes or cfDNA as a combined biomarker for the differential diagnosis of a solid cancer and a haematological cancer disease.

In one embodiment the cancer profile is identified as that of a haematological cancer, and not that of a solid cancer (or vice versa), by comparison to a similar profile generated for a known (or reference) haematological cancer sample with a highly elevated chromatin fragment concentration (for example as shown in FIG. 7B) and/or to a profile generated for a patient with a known (or reference) solid cancer disease with a highly elevated chromatin fragment concentration (for example as shown in FIG. 3B).

The cut-off values used for the purposes of the invention will vary with the chromatin fragment measurements made (for example quantification of cfDNA or nucleosomes will lead to different cut-off values) and vary with the method of measurement (for example different nucleosome assays or different DNA quantification methods). It will be understood that the cut-off selected for use herein at approximately 350 ng/ml H3.1-nucleosomes is for illustrative purposes and other cut-off values may be used.

We have shown that a sample containing circulating chromatin fragments can be used to differentially identify the presence of cancer or a NETosis related disease (for example an inflammatory disease such as sepsis) as well as for the differential diagnosis of a solid or liquid (haematological) tumour in a subject on the basis of the size distribution profile of chromatin fragments present.

It will be understood that the surprising finding that the size of circulating chromatin fragments can be used to distinguish cancer and NETosis related diseases also means that this information can be used to diagnose these diseases individually. Therefore, according to a further aspect, there is provided the use of a size profile of circulating chromatin fragments, cf-nucleosomes or cfDNA present in a blood, serum or plasma sample as a biomarker for the diagnosis, detection or monitoring of cancer.

According to a further aspect, there is provided the use of a size profile of circulating chromatin fragments, cf-nucleosomes or cfDNA present in a blood, serum or plasma sample as a biomarker for the diagnosis, detection or monitoring of a NETosis related disease.

The level of circulating chromatin fragments may be used in combination with methods of the invention to aid diagnosis. Therefore, in one embodiment the size profile is used in combination with the level of circulating chromatin fragments as a biomarker for the differential diagnosis of cancer or a NETosis related disease.

In a further embodiment the size profile is used in combination with the level of circulating chromatin fragments as a biomarker for the differential diagnosis of a solid and a haematological cancer and/or a NETosis related disease.

References herein to a “size profile” will be understood to mean the sizes of circulating chromatin fragments (which include cf-nucleosomes and cfDNA fragments) in a biological sample. For example, a size profile may be a histogram that provides a distribution of an amount of DNA fragments at a variety of sizes. It may also be referred to as the “size distribution profile”. The size profile may be measured using methods known in the art, for example, by DNA sequencing (e.g. paired-end massively parallel sequencing), by polymerase chain reaction (PCR), or by any physico-chemical method including, without limitation, spectrometry, chromatography or electrophoresis (e.g. using a Bioanalyzer). The latter example is particularly useful because electrophoresis using a Bioanalyzer is a quick and relatively cheap procedure. In one embodiment, the size profile is obtained by an electrophoresis method.

We have observed that circulating chromatin fragments, cf-nucleosomes or cfDNA levels may be elevated in early stage solid cancer disease, are commonly elevated in late stage solid cancer disease and may be highly elevated in early and late stage haematological cancer disease.

Therefore, according to a further aspect of the invention, there is provided a method for the differential diagnosis of cancer and a NETosis related disease in a subject which comprises the steps of:
(i) determining the size profile of circulating chromatin fragments in a blood, serum or plasma sample obtained from the subject, wherein the subject has been determined to have an elevated level of circulating chromatin fragments; and
(ii) using the size profile of the circulating chromatin fragments to indicate whether the disease present is cancer or a NETosis related disease,
   wherein the size profile of the circulating chromatin fragments present in a subject with a NETosis related disease is larger compared to a subject with cancer.

According to a further aspect of the invention, there is provided a method for determining the origin of a chromatin fragment in a blood, serum or plasma sample obtained from a subject which comprises the steps of:
(i) determining the size profile of circulating chromatin fragments in the blood, serum or plasma sample obtained from the subject; and
(ii) using the size profile of the circulating chromatin fragments to indicate the probable origin of the chromatin fragment. As described herein, the size profile of a circulating chromatin fragment derived from a NETosis process (e.g. cell death from NETosis, such as in a NETosis related disease) is larger compared to a circulating chromatin fragment derived from a non-NETosis process (e.g. apoptosis of a cancer cell).

References to determining the size profile of circulating chromatin fragments will be understood to include measuring the size profile of circulating chromatin fragments, cf-nucleosomes or cfDNA present in the sample, as described hereinbefore.

As described herein, the present invention is based upon the surprising finding that there is a difference in size between circulating chromatin fragments in samples obtained from a cancer patient compared to patients with a non-cancer, NETosis related disease. In particular, samples obtained from subjects with a NETosis related disease contain larger circulating chromatin fragments, e.g. fragments comprising more than about 800 bp, such as more than about 1000 bp. This may be shown, for example, by the widest and/or largest peak on a size distribution profile being at around 800 bp or more, indicating that most circulating chromatin fragments are circulating with oligonucleosomes (i.e. chains of multiple nucleosomes, usually at least 5 nucleosomes). In contrast, samples obtained from subjects with cancer contain smaller circulating chromatin fragments, e.g. fragments comprising 300 bp or less. This may be shown, for example, by the largest peak on a size distribution profile being at around 160 bp, indicating that most circulating chromatin fragments are circulating as mononucleosomes.

In one embodiment, the size profile of circulating chromatin fragments in a sample obtained from a subject with cancer contains the largest peak at about 160 bp, in particular between 140 bp and 300 bp. Peaks representing large chromatin fragments are either absent or very small indicating that the circulating chromatin fragments contain primarily mononucleosomes.

In one embodiment, the method additionally comprises measuring or detecting the level (or concentration) of circulating cell free nucleosomes in the blood, serum or plasma sample prior to step (i). In a further embodiment, said measurement or detection comprises an immunoassay, immunochemical, mass spectroscopy, chromatographic, chromatin immunoprecipitation or biosensor method.

In one embodiment, the size of the circulating chromatin fragments is used in combination with the level of circulating chromatin fragments to indicate whether the disease present is cancer or a NETosis related disease.

In one embodiment, the method additionally comprises extracting cfDNA from the circulating chromatin fragments in the sample and sequencing the extracted cfDNA. In a further embodiment, said sequencing comprises Next Generation Sequencing (NGS).

In one embodiment, the size of the circulating chromatin fragments is used in combination with analysis of the sequenced cfDNA to indicate whether the disease present is cancer or a NETosis related disease.

According to a further aspect, there is provided a method for the detection and differential diagnosis of cancer and a NETosis related disease in a subject which comprises the steps of:
(i) measuring or detecting the level of circulating chromatin fragments in a blood, serum or plasma sample obtained from the subject;
(ii) using the level of circulating chromatin fragments to indicate the presence of a NETosis related disease or cancer in the subject;
(iii) determining the size of the circulating chromatin fragments in the blood, serum or plasma sample; and
(iv) using the size of the circulating chromatin fragment determined in step (iii) to indicate whether the disease present is cancer or a NETosis related disease.

In some embodiments the circulating chromatin fragment is a cfDNA fragment. In some embodiments the circulating chromatin fragment is a cf-nucleosome.

In a preferred embodiment the circulating chromatin fragment measured in step (ii) is a cf-nucleosome and the circulating chromatin fragments whose size profile is determined in step (iii) are cfDNA fragments (optionally extracted from the cf-nucleosomes or the plasma sample obtained from the subject).

In one embodiment serum or plasma samples to be analysed may be centrifuged prior to analysis for removal of cellular debris or chromatin material contaminants.

As illustrated in the Figures herein, plasma samples obtained from subjects diagnosed with cancer or sepsis can be differentiated by any method for sizing chromatin fragments including, for example without limitation, by electrophoresis or polymerase chain reaction (PCR). It will be understood that any parameter of chromatin fragment size may be used for the purposes of the invention including, without limitation, the size distribution profile of chromatin fragments present, the level of large fragments present, the proportion of large fragments present, the mean fragment size present, the median fragment size present, the quantity or amount of fragments present that exceed a certain size, the quantity or amount of fragments present that are below a certain size, the relative proportions of fragments present that exceed a certain size compared to those that are below a (possibly different) certain size, and the size of the largest fragment present. Cut-off levels may be determined for size parameters so that different disease conditions can be classified as indicated to present above or below a cut-off. Many cut-offs in common use in pathology also include grey areas where a confident result cannot be determined and the test should be repeated. Such grey areas may also occur for the purposes of the current invention.

In one embodiment, there is provided a method for the detection and differential diagnosis of cancer and a NETosis related disease in a subject which comprises the steps of:
(i) measuring or detecting the level of circulating cell free nucleosomes in a blood, serum or plasma sample obtained from the subject;
(ii) using the level of cell free nucleosomes to indicate the presence of a NETosis related disease or cancer in the subject;
(iii) optionally extracting cfDNA from the blood, serum or plasma sample;
(iv) optionally preparing a cfDNA library for sequencing;
(v) determining the size of one or more cfDNA fragments present in, or extracted from, the sample; and
(vi) using the size of one or more cfDNA fragments determined in step (v) to indicate whether the disease present is cancer or a NETosis related disease.

We have shown that the level of circulating chromatin fragments may be used in combination with chromatin fragment size to differentiate samples obtained from a subject with cancer or from a subject with a NETosis related disease.

Therefore, in one embodiment, there is provided a method for the detection and differential diagnosis of cancer and a NETosis related disease in a subject which comprises the steps of:
(i) measuring or detecting the level of circulating chromatin fragments in a blood, serum or plasma sample obtained from the subject;
(ii) using the level of circulating chromatin fragments to indicate the presence of a NETosis related disease or cancer in the subject;
(iii) optionally extracting cfDNA from the blood, serum or plasma sample;
(iv) determining the size of one or more cfDNA fragments present in, or extracted from, the sample; and
(v) using the level of circulating chromatin fragments determined in step (i) in combination with the size of one or more cfDNA fragments determined in step (iv) to indicate whether the disease present is cancer or a NETosis related disease.

References to “subject” or “patient” are used interchangeably herein. The subject may be a human or an animal subject. In one embodiment the subject is a human subject. In some embodiments the subject is a (non-human) animal subject. In a further embodiment, the animal is a companion animal (also referred to as a pet or domestic animal). Companion animals include, for example dogs, cats, rabbits, ferrets, horses, cows, or the like. In particular, the companion animal is a dog or cat, particularly a dog. The methods described herein may be performed in vitro, or ex vivo.

### Quantification of Chromatin Fragments

The present invention is not limited to any particular method for quantifying circulating chromatin fragments and any suitable method may be used. Chromatin fragments, cfDNA or cf-nucleosomes may be measured by many methods including, for example without limitation, binding methods such as immunochemical or immunoassay methods or binding by DNA intercalating dyes, sequencing (for example to determine read numbers), rtPCR methods and spectroscopic methods.

In one embodiment, the size profile is obtained using a DNA intercalating dye. A DNA intercalating dye may be used to bind preferentially to chromatin fragments that contain linker DNA. Nucleosome bound DNA is relatively inaccessible to binding by DNA intercalating dyes. The unbound DNA between nucleosomes (linker DNA) is more strongly intercalated by DNA intercalating dyes (see for example, Bosire et al PLoS One 2019 doi:10.1371/journal.pone.0224936). The present authors have found that cell free mononucleosomes, of the size of tumour derived nucleosomes, typically bind low levels of fluorescence labelled DNA intercalating dyes and give low fluorescence signals. In contrast, larger chromatin fragments, e.g. the size of NETs and some NETs metabolites, typically bind higher levels of fluorescence labelled DNA intercalating dyes and give high fluorescence signals because they comprise much more linker DNA.

Therefore in one embodiment, there is provided a method for the detection and differential diagnosis of cancer and a NETosis related disease in a subject which comprises the steps of:
(i) measuring or detecting the level of circulating chromatin fragments in a blood, serum or plasma sample obtained from the subject;
(ii) using the level of circulating chromatin fragments to indicate the presence of a NETosis related disease or cancer in the subject;
(iii) determining the size of the circulating chromatin fragments in the blood, serum or plasma sample by contacting the sample with a DNA intercalating dye and determining the degree of binding of the DNA intercalating dye; and
(iv) using the size of the circulating chromatin fragment determined in step (iii) to indicate whether the disease present is cancer or a NETosis related disease.

As described herein, the size profile of the circulating chromatin fragments present in a subject with a NETosis related disease is larger compared to a subject with cancer. Therefore, in this embodiment of the invention, a larger degree of binding (e.g. a higher fluorescence) of the DNA intercalating dye will be measured for circulating chromatin fragments derived from a subject with a NETosis related disease compared to a subject with cancer.

DNA intercalation occurs when ligands of an appropriate size and chemical nature fit themselves in between base pairs of DNA. These ligands are mostly polycyclic, aromatic, and planar. A wide variety of intercalating dyes are used for binding to DNA including, without limitation, SYTOX™ Orange, SYTOX™ Green, PicoGreen®, SYBR Gold, YO-PRO-1, YOYO-1 and POPO-3. It will be understood that any DNA intercalating dye may be used for the purposes of the invention. In one embodiment, the DNA intercalating dye is labelled with a fluorescent label and the amount of fluorescence is used to measure the degree of binding. In some embodiments, the intercalating dye is SYTOX™ Green or PicoGreen® ((2-(n-bis-(3-dimethylaminopropyl)-amino)-4-(2,3-dihydro-3-methyl-(benzo-1,3-thiazol-2-yl)-methylidene)-1-phenyl-quinolinium)).

According to one aspect of the invention, there is provided a method for determining the (probable) origin of a circulating chromatin fragment in a blood, serum or plasma sample obtained from a subject which comprises the steps of:
(i) contacting the circulating chromatin fragment present in the sample with a DNA intercalating dye;
(ii) determining the degree of binding of the DNA intercalating dye to the circulating chromatin fragment; and
(iii) using the degree of binding of the DNA intercalating dye to the circulating chromatin fragment to indicate the (probable) origin of the circulating chromatin fragment.

The origin of the chromatin fragment may be NETosis or non-NETosis derived. As described herein, the size profile of a circulating chromatin fragment derived from a NETosis process (e.g. cell death from NETosis, such as in a NETosis related disease) is larger compared to a circulating chromatin fragment derived from a non-NETosis process (e.g. apoptosis of a cancer cell)

When a DNA intercalating dye is used, the method may additionally comprise contacting the sample with a binding agent which specifically binds to the circulating chromatin fragment (e.g. a binding agent which specifically binds to nucleosomes or a component thereof). This step may be performed before or after the DNA intercalating dye is applied. The binding agent directed to bind to a chromatin fragment is used to ensure that only the degree of binding detected for the DNA intercalating dye is associated with chromatin fragments and not other species which may be present in the sample.

The binding of the DNA intercalating dye to a chromatin fragment is detectable through the nature of the dye, or labelled dye, used. In some embodiments the DNA intercalating dye is coloured and measured by detecting ultraviolet (UV) or other visible methods. In some embodiments the DNA intercalating dye is fluorescent.

In a further aspect, there is provided a method for measuring nucleosomes in a sample which comprises the steps of:
(i) contacting the sample with a binding agent which specifically binds to a nucleosome or a component thereof;
(ii) contacting the nucleosomes bound in step (i) with a DNA intercalating dye;
(iii) determining the degree of binding of the DNA intercalating dye to the nucleosomes; and
(iv) using the degree of binding of the DNA intercalating dye to measure the amount of nucleosomes present in the sample.

According another aspect, there is provided a method for measuring nucleosomes in a sample which comprises the steps of:
(i) contacting the sample with a DNA intercalating dye;
(ii) contacting the sample with a binding agent which specifically binds to a nucleosome or a component thereof;
(iii) determining the degree of binding of the DNA intercalating dye to the nucleosomes; and
(iv) using the degree of binding of the DNA intercalating dye to measure the amount of nucleosomes present in the sample.

In one embodiment, the amount of nucleosomes is measured as the level or concentration of nucleosomes present in the sample.

In one embodiment, the nucleosomes measured are subsequently analysed, such as used the analysis methods described herein (e.g. mass spectrometry or DNA sequencing).

As described herein, the sample may be a blood, serum or plasma sample. The nucleosomes may therefore be referred to as cell free nucleosomes.

In some embodiments the binding agent is linked to a solid phase. Therefore, the circulating chromatin fragment (e.g. nucleosome) may be bound and isolated from the sample before analysis of the degree of DNA intercalating dye binding.

In preferred embodiments, the binding agent is an antibody.

As described herein, in preferred embodiments, the circulating chromatin fragment comprises a nucleosome. In one embodiment the binding agent specifically binds to nucleosomes or a component thereof. In one embodiment the binding agent is directed to bind to a post-translationally modification (PTM) of a histone. In one embodiment the binding agent is directed to a histone isoform. In one embodiment the binding agent is directed to bind to a nucleotide. In one embodiment the binding agent is directed to bind to a non-histone chromatin protein or protein adduct.

### Chromatin Fragment Size Profiling

Fragment size profiling may be performed by any suitable method. In some embodiments the method employed to determine the size profile of cfDNA fragments is electrophoresis. These methods are well known in the art and automated systems are available including, for example without limitation, electropherograms such as those shown here in FIGS. 2-6 produced using the Bioanalyzer System available from Agilent Technologies. Other electrophoresis systems available include the TapeStation System and the Fragment Analyzer System. The advantages of electrophoresis include rapidity and low cost.

DNA sequencing methods also provide fragment size and fragment size profile information. Therefore, in some embodiments the method employed to determine the size profile of cfDNA fragments is cfDNA sequencing. Any sequencing method may be employed. Although sequencing methods tend to be slower and higher cost than electrophoresis, the sizing of cfDNA by next generation DNA sequencing is more accurate than by electrophoresis. Next generation sequencing (also known as high-throughput sequencing) is any sequencing method that allows for rapid, high-throughput sequencing of base pairs from DNA or RNA samples. Such sequencing is well known in the art and can include, for example, Illumina arrays and ion torrent.

The most common next generation sequencing method involves sequencing by synthesis (SBS) wherein the addition of labelled nucleotides is tracked as the DNA chain is copied in a massively parallel fashion (for example as employed by the next generation DNA sequencing instruments available from Illumina). However, this is an expensive and slow method of obtaining cfDNA fragment size profiles requiring more than 1 day to complete. SBS sequencing involves the sequencing (only) of the ends of the DNA molecules of the input sample DNA. The length of the ends to be sequenced is determined in advance. A typical sequencing method might involve sequencing 200 bp at each end of the DNA molecules. The results are used to reconstruct the DNA molecules in silico providing information on the size of the fragments sequenced.

Determination of cfDNA fragment size using PCR methods is well described in the art. In brief, a cfDNA fragment will not be amplified if the target amplicon is larger than the fragment. For example, a 200 bp cfDNA fragment will not be amplified using PCR oligonucleotide primers targeted to a 500 bp sequence. Therefore a pair of primers targeting an amplicon of 100 bp may be used to amplify fragments of size ≥100 bp. Similarly, a pair of primers targeting an amplicon of 200 bp may be used to amplify fragments of size 2200 bp. The use of a number of such primer pairs targeted to amplicons of increasing size may be used to determine the size profile of cfDNA fragments in a sample (Sanchez et al. npj Genomic Med 3 (2018); doi.org/10.1038/s41525-018-0069-0).

Therefore, in one embodiment a PCR method is used to determine the size of one or more cfDNA fragments as a biomarker for the differential diagnosis of cancer or a NETosis related disease.

In one embodiment a numerical parameter or derivative of fragment size is used. It will be understood that any of a large variety of numerical parameters of fragment size distribution will be useful for methods of the invention. Examples include, without limitation, the level or concentration of large and/or mononucleosomes sized chromatin fragments present in a sample, the proportion of large and/or mononucleosomes sized fragments present, the median or mean fragment size present, the size of the largest fragment(s) present, the number or proportion of fragments in certain size ranges (for example 100-300 bp or >1000 bp) and many others. Further any units of numerical representation may be used including for example, without limitation, fragment numbers, fragment concentrations, Areas Under Curves (AUC) and any methodological output units (e.g. arbitrary units, optical density, fluorescence intensity, nephelometric units turbidimetry units and others).

In addition, any of these size distribution parameters may also be used in conjunction with the level of circulating chromatin fragments present to further differentiate between an inflammatory disease and a cancer with an inflammatory component.

### Additional Biomarkers

Chromatin released by neutrophils into the extracellular space as NETs may comprise additional proteins that are specific to NETs including, without limitation, myeloperoxidase (MPO) and neutrophil elastase (NE). These NETs specific proteins are not present in circulating chromatin fragments of other origins. In one embodiment the presence of circulating chromatin of a NETosis origin in a sample is determined by analysing the sample for one or more NETs specific proteins (for example without limitation MPO or NE).

The level of a NETs specific protein in a sample may be used in combination with either or both of the level of circulating chromatin fragments present and/or the size, or size profile, of chromatin fragments present in the sample to detect or diagnose a cancer disease or a NETosis related disease using methods of the invention.

Therefore, in one embodiment, there is provided a method for the detection and differential diagnosis of cancer and a NETosis related disease in a subject which comprises the steps of:
(i) measuring or detecting the level of circulating chromatin fragments in a blood, serum or plasma sample obtained from the subject;
(ii) using the level of circulating chromatin fragments to indicate the presence of a NETosis related disease or cancer in the subject;
(iii) measuring or detecting the level of a NETs specific protein in the blood, serum or plasma sample; and
(iv) using the level of the circulating chromatin fragments determined in step (i) in combination with the level of the NETs specific protein determined in step (iii) to indicate whether the disease present is cancer or a NETosis related disease.

In one embodiment the size profile is used in combination with the level of a NETs specific protein as a biomarker for the differential diagnosis of cancer or a NETosis related disease. Therefore, in one embodiment, there is provided a method for the detection and differential diagnosis of cancer and a NETosis related disease in a subject which comprises the steps of:
(i) determining the size of one or more cfDNA fragments present in, or extracted from, a blood, serum or plasma sample obtained from the subject;
(ii) measuring or detecting the level of a NETs specific protein in the blood, serum or plasma sample; and
(iii) using the size of one or more cfDNA fragments determined in step (i) in combination with the level of the NETs specific protein determined in step (ii) to detect the presence of a disease in the subject and to indicate whether the disease present is cancer or a NETosis related disease.

In one embodiment, there is provided a method for the detection and differential diagnosis of cancer and a NETosis related disease in a subject which comprises the steps of:
(i) measuring or detecting the level of circulating chromatin fragments in a blood, serum or plasma sample obtained from the subject;
(ii) using the level of circulating chromatin fragments to indicate the presence of a NETosis related disease or cancer in the subject;
(iii) measuring or detecting the level of a NETs specific protein in the blood, serum or plasma sample;
(iv) determining the size of one or more cfDNA fragments present in, or extracted from, a blood, serum or plasma sample obtained from the subject; and
(v) using the level of the circulating chromatin fragments determined in step (i) in combination with the level of the NETs specific protein determined in step (iii) and the size of one or more cfDNA fragments determined in step (iv) to indicate whether the disease present is cancer or a NETosis related disease.

The measurement of a NETS specific protein such as MPO or NE may be performed by any method. A common method is by immunoassay. For example, many commercial immunoassay kits are available for MPO and NE.

### NETosis Related Diseases

Inflammatory, autoimmune, thrombotic, vascular, neurological and many other conditions have a NETosis component or cause. The NETosis related conditions with the most serious, near term, acute sequelae are organ transplant rejection and SIRS related conditions including sepsis, COVID-19, influenza, ARDS, SARS, pneumonia and others involving a hyperimmune reaction, cytokine storm, organ failure and/or overproduction of NETs. It will be understood that the term “NETosis related disease” used herein, refers to a condition or disease wherein ETs or NETs contribute to pathogenesis, chronicity, or worsening of the disease.

Most subjects infected with bacterial infections or viral infections such as influenza or coronavirus experience mild illness. However, some population subgroups, including elderly persons aged over 60 years and persons with an underlying medical condition such as diabetes, chronic lung conditions and particularly chronic cardiac conditions, are at risk of severe effects including SIRS, ARDS, SARS, pneumonia and death. The exact mechanism by which influenza or coronavirus infection leads to complications including pneumonia is not clear, but it is thought to be caused by a hyperimmune reaction to the viral infection in which excessive NETs contribute to acute injury of the lung leading to pneumonia and, in the worst cases, death.

NETosis related diseases include, but are not limited to: infectious diseases; sepsis; systemic inflammatory response syndrome (SIRS); acute respiratory distress syndrome (ARDS); severe acute respiratory syndrome (SARS); acute lung injury (ALI); multi-organ failure or multi-organ dysfunction syndrome (MODS), e.g. from ARDS, haemorrhagic shock, surgery, burns, or sepsis; pneumonia; influenza; tuberculosis; infections; stroke; myocardial ischemia/infarction; coronary artery disease; acute coronary syndrome; heart failure; reperfusion injury; acute kidney injury (AKI); chronic kidney disease; diabetes, including type 1 or type 2 diabetes; angiopathies; vasculopathies; end-organ complications (e.g., retinopathy or diabetic kidney disease); deep vein thrombosis; atherosclerotic thrombosis; appendicitis; multiple sclerosis; systemic lupus erythematosus (SLE); lupus nephritis; rheumatoid arthritis; chronic obstructive pulmonary disease (COPD); cystic fibrosis; pulmonary disease; sickle cell disease; inflammatory bowel disease (IBD); Crohn's disease; ulcerative colitis; and indeterminate colitis. For the avoidance of doubt, NETosis related diseases as referred to herein do not include cancer (i.e. non-cancer NETosis related diseases).

### Sepsis

Sepsis is a severe inflammatory medical condition that can lead to hemodynamic shock and acute organ failure and is a leading cause of hospital mortality. Sepsis can involve any or all of low blood pressure, accelerated heart rate, pain, fever with sweaty skin and feeling cold, shortness of breath and disorientation or confusion. The condition of sepsis patients may deteriorate rapidly over hours into septic shock with low blood pressure, stroke, respiratory failure, heart failure, or multiple organ failure. Sepsis requires immediate treatment with intravenous fluids and antimicrobials often in an intensive care setting. Mechanical ventilation and dialysis may be needed to support the function of the lungs and kidneys, as well as preventive measures for thrombosis. Patient outcome depends on prompt diagnosis and early treatment. However, sepsis or SIRS is not easy to diagnose, especially in critically ill patients.

In sepsis, an inflammatory stimulus triggers a severe inflammatory response characterised by a cytokine storm with elevated levels of circulating cytokines as well as elevated production of NETs and elevated levels of circulating cf-nucleosomes and cfDNA. Elevated cytokine and NETs production by NETosis is pathological and has been found to lead to thrombosis, low blood pressure, high blood lactate and low urine output, leading eventually to respiratory distress, loss of consciousness and multiple organ failure.

Sepsis or SIRS may arise from non-infectious inflammatory stimuli such as polytrauma, surgery, pancreatitis or burns. More commonly, sepsis may be caused by infectious inflammatory stimuli such as bacterial, fungal, viral or protozoan infection. Influenza and COVID-19 are examples of well-known viral infections that can lead to SIRS.

Rapid early identification of these patients to enable timely treatment is important because their condition may deteriorate rapidly and failure to treat early may result in death.

### Infection

A primary role of NETs is to trap and kill pathogens locally and prevent spread of the infection around the body. Infection is therefore often associated with both inflammation, including NETosis, and cell death caused by the pathogen. Methods of the invention can be used to measure and distinguish nucleosomes derived both from NETosis (originating from rapidly dividing neutrophil cells) from nucleosomes derived from non-NETosis diseases, such as cancer. This will provide better and more complete information regarding the status of the subject and lead to an improved understanding of the subjects' clinical condition leading to better clinical management of infections of the lung, liver, kidney, heart, CNS and other organs.

In one embodiment, the NETosis related disease is an infection, i.e. an infection involving a hyperimmune reaction, cytokine storm, organ failure and/or overproduction of NETs. Infections that lead to sepsis most often start in the lung, urinary tract, skin, or gastrointestinal tract. Such infections include, but are not limited to, bacterial infections and viral infections. In one embodiment, the infection is a viral, bacterial, fungal or microbial infection.

Infection by gram-positive or gram-negative bacteria may lead to overproduction of NETs. Some of the most frequently isolated bacteria in sepsis are Staphylococcus aureus (S. aureus), Streptococcus pyogenes (S. pyogenes), Klebsiella spp., Escherichia coli (E. coli), Pseudomonas aeruginosa (P. aeruginosa) Mycobacterium tuberculosis and Haemophilus influenzae. Bacterial exotoxins, for example tetanus and diphtheria, may lead to systemic sepsis even if the organism itself remains localized.

In a further embodiment, the infection is a viral infection. Viral infections may include infections caused by respiratory syncytial virus (RSV), influenza type A, influenza type B and coronaviruses (e.g. COVID-19). Some viral infections that most frequently lead to overproduction of NETs include influenza and COVID-19.

The infection can be defined by the tissue affected by the disease. For example, the disease may affect the heart, brain, kidneys, liver, pancreas, lungs and/or blood and the infection may be a bacterial, viral, fungal or microbial infection known to commonly affect such tissues or organs. In one embodiment, the infection is a respiratory tract infection. According to this embodiment, the infection affects the lungs, upper and/or lower respiratory tract.

Other tissues which may be affected by the disease include peripheral tissues such as limbs, hands and feet and the infection may be a bacterial infection (e.g. gangrene). In one embodiment, the infection and/or disease may affect multiple tissues or organs simultaneously. For example, the infection may be a bacterial infection of a limb, hand or foot and the disease may also affect the blood (e.g. sepsis). In one embodiment, the infection is sepsis. In another example, the disease may be cardiac or coronary failure and other tissues or organs affected by the disease may include the kidneys and renal system and/or the brain (e.g. stroke). In a yet further example, the disease may affect the lungs or the infection may be a respiratory tract infection and other tissues or organs affected may include the heart, coronary system and/or brain (e.g. heart failure, myocardial infarction and/or stroke).

### Other NETosis Related Diseases

In another embodiment, the NETosis related disease is an inflammatory condition, i.e. an inflammatory condition involving a hyperimmune reaction, cytokine storm, organ failure and/or overproduction of NETs. Such inflammatory conditions include, but are not limited to, systemic lupus erythematosus, rheumatoid arthritis, cystic fibrosis, deep vein thrombosis and Crohn's disease.

In one embodiment, the NETosis related disease is selected from: sepsis, COVID-19, influenza, SIRS, ARDS, SARS and pneumonia. In a further embodiment, the NETosis related disease is sepsis.

### Cancers

In some embodiments the cancer is a haematological cancer including without limitation leukaemias, lymphomas (including canine lymphoma), myelomas and angiosarcomas (including canine hemangiosarcoma). In some embodiments the cancer disease is a solid cancer including without limitation, lung cancer, liver cancer, prostate cancer, breast cancer, gastric cancer, colorectal cancer, thyroid cancer, skin cancer, bladder cancer, cervical cancer, pancreatic cancer, endometrial cancer or renal cancer.

Haematological cancers are cancers of the blood, therefore may also be referred to as “liquid or blood cancers”. There are 3 principal types of haematological cancers: leukaemias, which are caused by the rapid production of abnormal white blood cells; lymphomas which are caused by abnormal lymphoma cells; and myelomas, which is a cancer of the plasma cells.

For the purposes of the present invention a blood cancer may be considered to be any cancer in direct contact with the circulation. This includes angiosarcomas and hemangiosarcomas as these are cancers of the vascular lining and share the proximity to the circulation of blood cell cancers.

Leukaemia is cancer of the blood cells which usually starts in the bone marrow and travels through the bloodstream. In leukaemia, the bone marrow produces mutated cells and spreads them into the blood, where they grow and crowd out healthy blood cells. Lymphoma diseases affect the cells in the lymphatic system. In lymphomas, immune cells called lymphocytes grow out of control and collect in lymph nodes, the spleen, in other lymph tissues or in neighbouring organs. Myeloma, also known as multiple myeloma, develops in the bone marrow and affects plasma cells, which produce antibodies that attack infections and diseases. Examples of blood cancers include Acute Lymphoblastic Leukaemia (ALL), Acute Myeloid Leukaemia (AML), Hodgkin Lymphoma (HL) and Non-Hodgkin Lymphoma (NHL).

References to “acute leukaemia” means the cancer progresses quickly and aggressively, usually requiring immediate treatment. ALL involves the development of large numbers of immature lymphocytes which are unable to fight infection. This causes the patient to have less room for healthy white blood cells, red blood cells, and platelets in the circulation. As a result, the patient usually suffers from a weakened immune system and the symptoms of anaemia, such as tiredness, breathlessness and an increased risk of excessive bleeding. The risk for developing ALL is highest in children younger than 5 years of age and it is the most common type of leukaemia that affects children. The risk then declines slowly until the mid-20s, and begins to rise again slowly after age 50. Overall, about 4 of every 10 cases of ALL are in adults.

AML affects myeloblasts which results in the accumulation of abnormal monocytes and granulocytes in the bone marrow. AML may also affect myeloid stem cells resulting in abnormal red blood cells or platelets. As with ALL, this causes the patient to have lower levels of healthy white blood cells, red blood cells, and platelets in the circulation. AML is one of the most common types of leukaemia in adults and the average age at diagnosis is 68.

HL and NHL are the two main types of lymphoma. HL has a particular appearance under the microscope and contains cells called Reed-Sternberg cells (a type of B lymphocyte that has become cancerous), whereas NHL looks different under the microscope and does not contain Reed-Sternberg cells. Most lymphomas are NHL and only about 1 in 5 are HL. NHL is a cancer affecting lymphocytes and usually starts in lymph nodes or lymph tissue. It is one of the more common cancers among children, teens and young adults.

Current methods of diagnosing leukaemia and myeloma involve obtaining a complete blood count (CBC) test to identify abnormal levels of white blood cells relative to red blood cells and platelets. However, an elevated white blood cell count (WBC) is not specific to patients with a haematological malignancy; it can also be the result of an ongoing response to infection or other inflammatory process. For lymphoma, an X-ray, CT or PET scan can be used to detect swollen lymph nodes, however this is also non-specific.

In order to confirm a diagnosis of a haematological cancer, a bone marrow or lymph node biopsy is required. Therefore overdiagnosis of haematological cancers at an early stage in the diagnostic process can lead to unnecessary biopsies which are invasive, potentially hazardous and relatively costly to healthcare providers. Cytogenetics analysis and/or immunophenotyping can also be used to confirm a haematological cancer diagnosis, however these methods are expensive to perform and therefore are typically only used at a late stage of the diagnostic process.

The diagnosis of solid and liquid cancers requires an invasive tissue biopsy. Therefore methods of the invention as described herein, may be used for the selection of subjects for whom a biopsy is required.

The epigenetic composition of circulating cell free nucleosomes in terms of their histone modification, histone variant, DNA modification and adduct content have also been investigated as blood based biomarkers in cancer, see WO 2005/019826, WO 2013/030577, WO 2013/030579 and WO 2013/084002.

There remains a need in the art to provide simple, cost-effective diagnostic methods for haematological cancers, especially those able to distinguish from patients with NETosis related diseases, but who may present with similar symptoms.

In one embodiment the haematological cancer is selected from lymphoma, leukaemia, myeloma, chronic myeloproliferative disease, monoclonal gammopathy of uncertain significance, myelodysplastic syndrome and amyloidosis. In a further embodiment, the haematological cancer is selected from leukaemia or lymphoma.

Leukaemia affects white blood cells and can be classified by the type of white cell affected (myeloid or lymphatic) and by the way the disease progresses (acute or chronic). Several types of leukaemia have been identified including, but not limited to: Acute Lymphoblastic Leukaemia (ALL; which may also be referred to as Acute Lymphocytic Leukaemia), Acute Myeloid Leukaemia (AML), Acute Megakaryoblastic Leukaemia (AMKL), Acute Promyelocytic Leukaemia (APL), Childhood Acute Myeloid Leukaemia (C-AML), Childhood Acute Lymphocytic Leukaemia (C-ALL), Chronic Eosinophilic Leukaemia (CEL), Chronic Lymphocytic Leukaemia (CLL), Chronic Myeloid Leukaemia (CML), Chronic Myelomonocytic Leukaemia (CMML), Chronic Neutrophilic Leukaemia, Hairy Cell Leukaemia, Juvenile Myelomonocytic Leukaemia (JMML), Large Granular Lymphocytic Leukaemia (LGLL), T-cell Acute Lymphoblastic Leukaemia and Prolymphocytic Leukaemia.

In one embodiment, the leukaemia is an acute leukaemia, such as Acute Lymphocytic Leukaemia (ALL), Acute Myeloid Leukaemia (AML), Acute Megakaryoblastic Leukaemia (AMKL) or Acute Promyelocytic Leukaemia (APL). In a further embodiment, the leukaemia is selected from Acute Lymphocytic Leukaemia (ALL) and Acute Myeloid Leukaemia (AML).

Alternatively, in one embodiment, the leukaemia is a chronic leukaemia, such as Chronic Eosinophilic Leukaemia (CEL), Chronic Lymphocytic Leukaemia (CLL), Chronic Myeloid Leukaemia (CML), Chronic Myelomonocytic Leukaemia (CMML) or Chronic Neutrophilic Leukaemia.

Both Hodgkin Lymphoma (HL) and Non-Hodgkin Lymphoma (NHL) are lymphomas. The majority of NHL patients are over the age of 55 when first diagnosed, whereas the median age for diagnosis of Hodgkin lymphoma is 39. In one embodiment, the lymphoma is Non-Hodgkin Lymphoma (NHL). NHL may arise in lymph nodes anywhere in the body, whereas HL typically begins in the upper body, such as the neck, chest or armpits.

Hodgkin lymphoma is often diagnosed at an early stage and is therefore considered one of the most treatable cancers. Non-Hodgkin lymphoma is typically not diagnosed until it has reached a more advanced stage, therefore methods of the invention find particular use in the diagnosis of NHL where there is a need to detect patients at an early stage of disease to improve treatment outcome. AML is the most common leukaemia in elderly subjects. AML progresses rapidly and requires early diagnosis for timely treatment. Therefore, the detection of AML is also an important application of methods of the invention.

### Circulating Chromatin Fragments

Uses and methods of the invention relate to measuring the size profile of circulating chromatin fragments present in the blood, serum or plasma sample. In one embodiment, the size profile of circulating chromatin fragments is measured in uses and methods of the invention. In an alternative embodiment, the size profile of cf-nucleosomes is measured in uses and methods of the invention. In an alternative embodiment, the size profile of cfDNA is measured in uses and methods of the invention.

The term “chromatin fragment” as used herein refers to a complex of proteins and nucleic acid whose origin lies in the chromosome or mitochondria of a cell. The term encompasses chromatin fragments found outside of cells, which may also be referred to as “cell free chromatin fragments”. A fragment of chromatin may contain a nucleosome and/or associated DNA and/or any of a huge variety of non-histone chromatin associated proteins in a multi-protein-nucleic acid complex. Some examples of non-histone chromatin associated proteins include transcription factors, cofactors, co-activators, co-repressors, RNA polymerase moieties, elongation factors, chromatin remodelling factors, mediators, STAT moieties, upstream binding factor (UBF) and others.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller (2007) Methods Mol. Biol. 361: 25-62).

NETs and ETs are chromatin fragments are released as long strings of nucleosomes.

References to “nucleosome” may refer to “cell free nucleosome” when detected in body fluid samples. It will be appreciated that the term cell free nucleosome throughout this document is intended to include any circulating chromatin fragment that includes one or more nucleosomes. “Epigenetic features”, “epigenetic signal features” or “epigenetic signal structures” of a cell free nucleosome as referred herein may comprise, without limitation, one or more histone post-translational modifications, histone isoforms, modified nucleotides and/or proteins bound to a nucleosome in a nucleosome-protein adduct.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

In one embodiment, the component of the nucleosome is a histone protein. References herein to “histone” refer to histones and modifications thereof, as described herein (e.g. post-translational modifications, mutations, isoforms, variants and fragments of histones, such as clipped histones).

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells and also form the repeating units present in ETs and NETs.

Normal cell turnover in adult humans involves the creation by cell division of a large number of cells daily and the death of a similar number, mainly by apoptosis but also by other cell death mechanisms including NETosis. Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenreider & Stieber (2009) Crit Rev Clin Lab Sci, 46(1): 1-24).

Mononucleosomes and oligonucleosomes can be detected by Enzyme-Linked ImmunoSorbant Assay (ELISA) and several methods have been reported (e.g. Salgame et al. (1997); Holdenrieder et al. (2001); van Nieuwenhuijze et al. (2003)). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts (for example myeloperoxidase, neutrophil elastase adducts or other adducts associated with NETs). Assays for these types of chromatin fragments are known in the art (for example, see WO 2005/019826, WO 2013/030579, WO 2013/030578, WO 2013/084002 which are herein incorporated by reference).

The biomarker used in the uses and methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone variant, a particular nucleotide and a protein adduct. In one embodiment, the epigenetic feature of the nucleosome is the histone isoform H3.1.

The structure of a nucleosome may vary by the inclusion of alternative histone isoforms or variants which are different gene or splice products and have different amino acid sequences. In one embodiment, the epigenetic feature of the nucleosome comprises a histone variant or isoform. It will be understood that the term “histone variant” and “histone isoform” may be used interchangeably herein. Many histone isoforms are known in the art. Histone isoforms can be classed into a number of families which are subdivided into individual types. The sequences of a large number of histone isoforms are known and publicly available for example in the National Human Genome Research Institute NHGRI Histone Database (Marino-Ramirez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, isoforms of histone H2 include H2A1, H2A2, mH2A1, mH2A2, H2AX and H2AZ. In another example, histone isoforms of H3 include H3.1, H3.2 and H3t. In one embodiment, the histone isoform is H3.1.

Another way the structure of nucleosomes may vary is by mutation. Therefore, in one embodiment, the epigenetic feature is a mutated histone. In a further embodiment, the mutation is in histone 3 (H3). In a yet further embodiment, the mutation in H3 is when lysine 27 is replaced by a methionine (H3K27M).

The structure of nucleosomes can vary by post translational modification (PTM) of histone proteins. PTM of histone proteins typically occurs on the tails of the core histones and common modifications include acetylation, methylation or ubiquitination of lysine residues as well as citrullination or methylation of arginine residues and phosphorylation of serine residues and many others. It will be understood that a histone PTM may occur on different isoforms (variants) of the histone. For example, the lysine residues that occur on the tail of histone H3 isoforms H3.1, H3.2 and H3.3 may be modified by acetylation or methylation. Many histone modifications are known in the art and the number is increasing as new modifications are identified (e.g. see Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be present on a core nucleosome histone (e.g. H2A, H2B, H3 or H4), or a linker histone (e.g. H1 or H5). Examples of PTMs are described in WO 2005/019826 and WO 2017/068359.

In one embodiment, the histone PTMs are selected from acetylation, methylation (which may be mono-, di- or tri-methylation), phosphorylation, ribosylation, citrullination, ubiquitination, hydroxylation, glycosylation, nitrosylation, glutamination and isomerisation. In one embodiment, the histone PTM is methylation of a lysine residue. In a further embodiment, the methylation is of a histone 3 lysine residue. In a yet further embodiment, the histone PTM is selected from H3K4Me, H3K4Me2, H3K9Me, H3K9Me3, H3K27Me3 or H3K36Me3. In one embodiment, the histone PTM is acetylation of a lysine residue. In a further embodiment, the acetylation is of a histone 3 lysine residue. In a yet further embodiment, the histone PTM is selected from H3K9Ac, H3K14Ac, H3K18Ac or H3K27Ac. In another embodiment, the histone PTM is H4PanAc. In one embodiment, the histone PTM is phosphorylation of a serine residue. In a further embodiment, the phosphorylation is of an isoform X of histone 2A (H2AX) serine residue or phosphorylation of a histone 3 serine residue. In a yet further embodiment, the histone PTM is selected from pH2AX or H3S10Ph. In one embodiment, the histone PTM is selected from citrullination or ribosylation. In a further embodiment, the histone PTM is citrullinated H3 (H3cit) or citrullinated H4 (H4cit). In a further embodiment, the histone PTM is citrullination of a histone 3 arginine residue. In a yet further embodiment, the histone PTM is H3R8Cit. In one embodiment, the histone PTM is selected from the group consisting of: H3K4Me, H3K4Me2, H3K9Me, H3K9Me3, H3K27Me3, H3K36Me3, H3K9Ac, H3K14Ac, H3K18Ac, H3K27Ac, H4PanAc, pH2AX, H3S10Ph and H3R8Cit.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes and without limitation, anti-pan-acetylation antibodies (e.g. a Pan-acetyl H4 antibody [H4panAc]), anti-citrullination antibodies or anti-ubiquitin antibodies.

In one embodiment, the epigenetic feature is a DNA modification. In addition to the epigenetic signalling mediated by nucleosome histone isoform and PTM composition, nucleosomes also differ in their nucleotide and modified nucleotide composition. Some nucleosomes may comprise more 5-methylcytosine residues, or 5-hydroxymethylcytosine residues or other nucleotides or modified nucleotides, than other nucleosomes. In one embodiment, the epigenetic feature is a DNA modification selected from 5-methylcytosine or 5-hydroxymethylcytosine. Thus, in some embodiments, the defined calibrated DNA modification is 5-methylcytosine or 5-hydroxymethylcytosine.

A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art. Therefore, in one embodiment, the epigenetic feature comprises one or more protein-nucleosome adducts or complexes.

It will be understood that more than one epigenetic feature of cell free nucleosomes may be detected in methods and uses of the invention. Multiple biomarkers may be used as a combined biomarker. Therefore, in one embodiment, the use comprises more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected. In one aspect, the use comprises a post-translational histone modification and a nucleosome adduct as a combined biomarker in a plasma sample, for the diagnosis or detection of NETosis related condition or cancer.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

As described herein, the method may additionally comprise measuring or detecting the level of circulating cell free nucleosomes. Said measurement or detection comprises methods described hereinbefore, such as an immunoassay, immunochemical, mass spectroscopy, chromatographic, chromatin immunoprecipitation or biosensor method. In one embodiment, the measurement or detection employs a single binding agent. In an alternative embodiment, the measurement or detection comprises a 2-site immunometric assay employing two binding agents.

Methods and uses described herein may be tested in body fluid samples, in particular blood, serum or plasma samples. Preferably, plasma samples are used. Plasma samples may be collected in collection tubes containing one or more anticoagulants such as ethylenediamine tetraacetic acid (EDTA), heparin, or sodium citrate, in particular EDTA.

It will be clear to those skilled in the art that the terms “antibody”, “binder” or “ligand” as used herein are not limiting but are intended to include any binder capable of specifically binding to particular molecules or entities and that any suitable binder can be used in the method of the invention. In one embodiment, the binding agent is an antibody. In an alternative embodiment, the binding agent is a chromatin binding protein. It will also be clear that the term “nucleosomes” is intended to include mononucleosomes and oligonucleosomes and any protein-DNA chromatin fragments that can be analysed in fluid media.

Methods of detecting biomarkers are known in the art. The most commonly used epitope binders in the art are antibodies or derivatives of an antibody that contain a specific binding domain. The antibody may be a polyclonal antibody or a monoclonal antibody or a fragment thereof capable of specific binding to the epitope. However, any binder capable of binding to a particular epitope may be used for the purposes of the invention. The reagents may comprise one or more ligands or binders, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc. The terms antibody or binder as used herein are interchangeable and refer to any moiety capable of specific binding to an epitope.

In one embodiment, the binding agent is directed to a histone, nucleosome core protein, DNA epitope or a protein adducted to a nucleosome. In a further embodiment, the binding agent is directed to a histone isoform, such as a histone isoform of a core histone, in particular a histone H3 isoform. In a particular embodiment, the binding agent specifically binds to histone isoform H3.1.

A binding agent is considered to “specifically bind” if there is a greater than 10 fold difference, and preferably a 25, 50 or 100 fold difference between the binding of the agent to a particular target epitope compared to an non-target epitope.

The binding agent may comprise an MHC molecule or part thereof which comprises the peptide binding groove. Alternatively the agent may comprise an anti-peptide antibody. As used herein, “antibody” includes a whole immunoglobulin molecule or a part thereof or a bioisostere or a mimetic thereof or a derivative thereof or a combination thereof. Examples of a part thereof include: Fab, F(ab)′2; and Fv. Examples of a bioisostere include single chain Fv (scFv) fragments, chimeric antibodies, bifunctional antibodies.

The term “mimetic” relates to any chemical which may be a peptide, polypeptide, antibody or other organic chemical which has the same binding specificity as the antibody.

The term “derivative” as used herein in relation to antibodies includes chemical modification of an antibody. Illustrative of such modifications would be replacement of hydrogen by an alkyl, acyl, or amino group.

The binding agent may be an aptamer or a non- immunoglobulin scaffold such as an affibody, an affilin molecule, an AdNectin, a lipocalin mutein, a DARPin, a Knottin, a Kunitz-type domain, an Avimer, a Tetranectin or a transbody.

In one embodiment, the method of measuring the level of nucleosomes comprises contacting the sample with a solid phase comprising a binding agent that detects nucleosomes or a component thereof, and detecting binding to said binding agent.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission.

In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

A change in the level of the biomarker in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment the monitoring of more rapid changes due to fast acting therapies may be conducted at shorter intervals of hours or days.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

Suitably, biosensors for detection of one or more biomarkers combine biomolecular recognition with appropriate means to convert detection of the presence, or quantitation, of the biomarker in the sample into a signal. Biosensors can be adapted for “alternate site” diagnostic testing, e.g. in the ward, outsubjects' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers.

Biomarkers for detecting the presence of a disease are essential targets for discovery of novel targets and drug molecules that retard or halt progression of the disorder. As the level of the biomarker is indicative of disorder and of drug response, the biomarker is useful for identification of novel therapeutic compounds in in vitro and/or in vivo assays. Biomarkers described herein can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

In a further aspect of the invention there is provided an instrument which quantifies circulating chromatin fragments and performs a fragment size analysis and interpolates the results thereof to provide a clinical result or output, for example related to the probability of cancer or NETosis related condition in a subject.

Identifying, detecting and/or quantifying can be performed by any method suitable to identify the presence and/or amount of a specific protein in a biological sample from a subject or a purification or extract of a biological sample or a dilution thereof. In particular, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner. The samples may be centrifuged prior to analysis for removal of cellular debris which may be contaminated with chromatin material. The present invention finds particular use in plasma samples which may be obtained from the subject.

Identification, detection and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying chromatin fragments can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

In one embodiment the methods of the present invention are used to detect circulating chromatin fragments in the circulation of organ or tissue transplant recipients and to characterise the fragments as having a NETosis origin. The presence of circulating chromatin fragments of NETs origin in these patients may be used as an early indicator of organ or tissue rejection. The methods of the invention may therefore be used to identify patients in need of stronger or weaker immunosuppression and used to determine and personalise drug regimens for each patient.

In one embodiment, the subject is suspected of relapse to a cancer. Minimal residual disease (MRD) is the name given to small numbers of cancer cells that remain in the person during treatment, or after treatment when the patient is in remission (i.e. patients with no symptoms or signs of disease). However, MRD is the major cause of relapse in cancer. Methods of the invention are therefore useful in monitoring patients who are suspected of relapse, particularly patients who are in remission from cancer.

The subject tested using the methods described herein may present with symptoms indicative of cancer, for example the symptoms of a haematological cancer may include anaemia, leucocytosis and/or swollen lymph nodes. In one embodiment, the subject has a high level of leucocytosis. This may also be referred to a “high white blood cell count”. Haematological cancers typically cause increased proliferation of abnormal white or red blood cells which results in a high white blood cell count. However, leucocytosis is not sufficient to diagnose a patient with a haematological cancer (in particular leukaemia) because it is frequently a sign of an inflammatory response, most commonly the result of infection. Therefore, methods of the invention are able to provide a more specific differential method to identify patients who are likely to be suffering from cancer or an inflammatory condition.

Detecting and/or quantifying may be compared to a cut-off level. Cut-off values can be predetermined by analysing results from multiple patients and controls, and determining a suitable value for classifying a subject as with or without the disease. For example, for diseases where the level of biomarker is higher in patients suffering from the disease, then if the level detected is higher than the cut-off, the patient is indicated to suffer from the disease. Alternatively, for diseases where the level of biomarker is lower in patients suffering from the disease, then if the level detected is lower than the cut-off, the patient is indicated to suffer from the disease. The advantages of using simple cut-off values include the ease with which clinicians are able to understand the test and the elimination of any need for software or other aids in the interpretation of the test results. Cut-off levels can be determined using methods in the art.

Detecting and/or quantifying may also be compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject, a non-diseased subject and/or a subject without a haematological cancer. Comparison with a control is well known in the field of diagnostics.

Therefore, in one embodiment, the method additionally comprises comparing the level of circulating chromatin fragments and the size profile of circulating chromatin fragments in a blood, serum or plasma sample taken from a subject with one or more controls. For example, the method may comprise comparing the level and sizes of cell free fragments present in a sample obtained from the subject with those present in a sample obtained from a normal subject, a subject with cancer or a subject with a NETosis related condition.

The control may be a subject with cancer for the differential diagnosis of a test subject suspected of a NETosis related disease such as sepsis. Similarly, the control may be a subject with a NETosis related disease such as sepsis for the differential diagnosis of a test subject suspected of cancer. The data provided herein shows that biomarkers of the invention were significantly different in cancer patients compared to patients with NETosis related diseases, therefore they may be used to differentially diagnose patients with cancers over patients with NETosis related diseases or vice versa. Both positive and negative controls may be used. Thus, the presence of a cancer disease in a subject may be confirmed by comparison of results with known cancer controls (positive control) as well as with known disease free or non-cancer controls (negative control). Similarly, the presence of a NETosis related disease in a subject may be confirmed by comparison of results with known NETosis related controls (positive control) as well as with known disease free or cancer controls (negative control).

In one embodiment, the level of cell free nucleosomes is elevated compared to the control. For example, the level of cell free nucleosomes may be more than about 100 ng/ml, such as more than about 350 ng/ml.

In one embodiment, the size of a circulating chromatin fragment, or the size profile of chromatin fragments, is compared to that of a control subject.

It will be understood that it is not necessary to measure control levels or size profiles for comparative purposes on every occasion. For example, for healthy/non-diseased controls, once the ‘normal range’ is established it can be used as a benchmark for all subsequent tests. A normal range can be established by obtaining samples from multiple control subjects without cancer or NETosis related disease and testing for the level of biomarker. Results for subjects suspected to have cancer or NETosis related condition can then be examined to see if they fall within, or outside of, the respective normal range. Use of a ‘normal range’ is standard practice for the detection of disease. Similarly, chromatin fragment size ranges or profiles or size cut-offs can be established for cancer and NETosis related conditions. Results for subjects suspected to have cancer or NETosis related disease can then similarly be examined to see if they fall within, or outside of, the respective ranges or cut-offs.

In one embodiment, the method additionally comprises determining at least one NETs specific protein. This level of NETs specific protein can be used in the interpretation of results.

In one embodiment, the method additionally comprises determining at least one clinical parameter for the patient. This parameter can be used in the interpretation of results. Clinical parameters may include any relevant clinical information for example, without limitation, gender, weight, Body Mass Index (BMI), smoking status, temperature and dietary habits. Therefore, in one embodiment, the clinical parameter is selected from the group consisting of: age, sex and body mass index (BMI).

In one embodiment, the method of the invention is performed to identify a subject at high risk of having a haematological cancer and therefore in need of further testing (i.e. further cancer investigations). The further testing may involve one or more of: biopsy (such as bone marrow biopsy or lymph node biopsy), cytogenetic testing, immunophenotyping, CT scanning, X-ray (in particular chest X-ray to identify swollen lymph nodes) and/or lumbar puncture.

Methods and biomarkers described herein may be used to identify if a patient is in need of a biopsy, in particular a bone marrow or lymph node biopsy (e.g. for patients with suspected haematological cancer). Therefore, according to a further aspect of the invention there is provided a method of identifying a patient in need of a biopsy comprising obtaining a blood, serum or plasma sample from said patient, detecting the level of cfDNA or cf-nucleosomes or other chromatin fragments in the sample, detecting the size profile of circulating chromatin fragments present in the sample and using the results obtained to identify whether the patient is in need of a biopsy. As described hereinbefore, when the size profile of circulating chromatin fragments is small, e.g. around 160 bp, this indicates that the circulating chromatin fragments are predominately present as mononucleosomes which is indicative of a patient with cancer and therefore in need of a biopsy to confirm a cancer diagnosis.

### Methods of Treatment

According to a further aspect, there is provided a method of treating cancer in a subject, which comprises the following steps:
(i) determining the size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is cancer; wherein the size profile of the circulating chromatin fragments in a subject with cancer is smaller compared to a subject with a NETosis related disease; and
(iii) administering a treatment to the subject if they are determined to have cancer in step (ii).

According to a further aspect, there is provided a method of treating a haematological cancer in a subject, which comprises the following steps:
(i) determining the size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is a haematological cancer; wherein the size profile of the circulating chromatin fragments in a subject with a haematological cancer is smaller compared to a subject with a NETosis related disease; and
(iii) administering a treatment to the subject if they are determined to have a haematological cancer in step (ii).

According to a further aspect, there is provided a method of treating a haematological cancer in a subject in need thereof, which comprises the step of administering a therapeutic agent to a subject identified as having a smaller size profile of circulating chromatin fragments in a blood, serum or plasma sample obtained from said subject, when compared to the size profile of circulating chromatin fragments in a blood, serum or plasma sample obtained from a subject with a NETosis related disease.

Suitable treatment methods may be determined by a trained physician. In one embodiment, the treatment is selected from one or more of: chemotherapy, immunotherapy, hormone therapy, biological therapy, radiotherapy, leukapheresis and stem cell transplant.

According to a further aspect, there is provided a method of treating a NETosis related disease in a subject, which comprises the following steps:
(i) determining the size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is a NETosis related disease; wherein the size profile of the circulating chromatin fragments in a subject with a NETosis related disease is larger compared to a subject with cancer; and
(iii) administering a treatment to the subject if they are determined to have a NETosis related disease in step (ii).

According to a further aspect, there is provided a method of treating a NETosis related disease in a subject in need thereof, which comprises the step of administering a therapy (e.g. a therapeutic agent) to a subject identified as having a larger size profile of circulating chromatin fragments in a blood, serum or plasma sample obtained from said subject, when compared to the size profile of circulating chromatin fragments in a blood, serum or plasma sample obtained from a subject with cancer. The therapy may include one or more suitable treatments for the condition including without limitation, drugs (e.g. anti-inflammatory drugs, blood thinning or clotting inhibitor drugs, therapeutic anti-NETs antibody drugs, DNase drugs, NETosis inhibitor drugs, anti-bacterial drugs or anti-viral drugs), apheresis treatments, ventilator support, fluid support or others.

In one embodiment, the treatment is selected from one or more of: antibiotic treatments (e.g. penicillins, cephalosporins, tetracyclines, aminoglycosides, macrolides, clindamycin, sulphonamides, trimethoprim, metronidazole, tinidazole, quinolones and/or nitrofurantoin), anti-microbial treatments (e.g. ethambutol, isoniazid, pyrazinamide, rifampicin, aminoglycosides (amikacin, kanamycin), polypeptides (capreomycin, viomycin, enviomycin), fluoroquinolones (ciprofloxacin, levofloxacin, moxifloxacin), thioamides (ethionamide, prothionamide), cycloserine (closerin), terizidone, rifabutin, macrolides (clarithromycin), linezolid, thioacetazone, thioridazine, arginine, vitamin D and/or R207910), anti-viral COVID treatments (e.g. remdesivir), anti-viral influenza treatments (e.g. amantadine, umifenovir, moroxydine, rimantadine, umifenovir, zanamivir and neuraminidase inhibitors, cap-dependent endonuclease inhibitors, adamantanes, peramivir, zanamivir, oseltamivir phosphate and baloxavir marboxil) as well as anti-viral treatments for other viral diseases that may lead to a high level of NETosis and anti-fungal treatments (e.g. clotrimazole, econazole, miconazole, terbinafine, fluconazole, ketoconazole and amphotericin).

In one embodiment, the treatment is an anti-inflammatory drug. Many steroidal and non-steroidal anti-inflammatory drugs are known in the art. Some examples of steroidal anti-inflammatory drugs include without limitation, dexamethasone, hydrocortisone, cortisone, betamethasone, prednisone, prednisolone, triamcinolone and methylprednisolone. Some examples of non-steroidal anti-inflammatory drugs include without limitation, aspirin, celecoxib, diclofenac, diflunisal, etodolac, ibuprofen, indomethacin, CD24Fc (CD24 protein attached to the Fc region of immunoglobulin G) and EXO-CD24 (CD24-Exosomes).

In one embodiment, treatment is a DNase treatment to digest excess NETs or an inhibitor of NETosis, such as an anthracycline drug. In a further embodiment, the anthracycline drug is selected from: epirubicin, daunorubicin, doxorubicin and idarubicin.

In one embodiment the treatment is a therapeutic antibody drug directed to bind to NETs or to a component part of a NET including, without limitation, a therapeutic antibody directed to bind to a nucleosome, or to any component part of a nucleosome. Examples include therapeutic antibodies directed to bind to nucleosomes containing histone isoform H3.1, citrullinated histones, myeloperoxidase, neutrophil elastase or C-Reactive Protein.

In one embodiment the treatment is a nucleic acid scavenger that adsorbs and/or removes nucleic acids from the circulation or from the body, for example the DNA scavenger polyamidoamine.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Example 1

Plasma samples were taken from a cohort of subjects diagnosed with a variety of solid and haematological cancers and from patients hospitalised for moderate COVID-19 infection. For each subject, a whole blood draw was collected in an EDTA vacutainer plasma BCT. The whole blood was centrifuged and plasma was transferred to a cryotube and frozen. The samples were analysed for intact cf-nucleosomes containing histone isoform H3.1 (H3.1-nucleosomes) by ELISA. The results are shown in FIG. 1. The results show that cf-nucleosomes and cfDNA levels may be elevated in samples taken from both patients with cancer and NETosis related diseases (i.e. COVID-19).

### Example 2

NETosis may be triggered by a number of stimuli including the presence of pathogens and coagulation. We used coagulation as a trigger to generate NETs in whole blood in order to investigate the circulating chromatin fragment profile produced by NETosis in blood in the absence of any cancer.

We generated NETs ex vivo in blood collected from 3 healthy subjects. To do this we collected whole blood from each healthy subject into 3 serum blood collection tubes (BCTs). The serum BCT tubes containing whole blood were left at room temperature for 30 minutes, 24 hours and 48 hours before processing by centrifugation at 3000×g for 10 minutes and the supernatant serum was removed into a cryotube and frozen at −80° C. until analysed. DNA was extracted from the serum samples using a commercial DNA extraction kit and the cfDNA fragment size profile was determined by electrophoresis using the Bioanalyzer system available from Agilent Technologies following the manufacturer's instructions. The chromatin fragment size profiles obtained as electropherograms are shown in FIG. 2.

The tube centrifuged at 30 minutes contained primarily the chromatin fragments circulating in vivo in the patient at blood draw. These fragments comprised predominantly mononucleosomes as shown by a single peak at approximately 180 bp. The results for EDTA plasma samples taken from healthy subjects (in which no NETosis is triggered) show a similar profile. In contrast, the tubes centrifuged at 24 and 48 hours contained large quantities of large chromatin fragments comprising up to 10,000 bp as well as greatly increased peaks relating to mononucleosomes (approx. 180 bp), di-nucleosomes (approx. 360 bp) and tri-nucleosomes (approx. 550 bp). The presence of large chromatin fragments in the samples in which NETosis was triggered and allowed to proceed for 24 or 48 hours is indicative of a NETosis origin for these nucleosomes.

We used these profiles as examples of chromatin fragment size profiles characteristic of profiles of a NETosis origin.

### Example 3

We investigated the circulating chromatin fragment size distribution in EDTA plasma samples obtained from 8 patients diagnosed with a variety of different solid cancers. EDTA plasma samples were used as the lack of a NETosis trigger in EDTA BCTs means that the cf-nucleosomes observed in the samples are representative of the circulating cf-nucleosomes.

We measured the H3.1-nucleosome level in the samples by immunoassay (e.g. see methods described in WO2016067029). The levels measured ranged from 101 to 642 ng/ml. DNA was extracted from the plasma samples using a commercial DNA extraction kit and the cfDNA fragment size profile was determined by electrophoresis using the Bioanalyzer system available from Agilent Technologies following the manufacturer's instructions. The profiles obtained are shown in FIG. 3.

We observed that profiles for some solid cancer samples contained peaks corresponding only or predominantly to mononucleosomes and oligonucleosomes with no peak, or very small peaks corresponding to large chromatin fragments. However, other solid cancer samples contained both peaks corresponding to mononucleosomes and oligonucleosomes as well as peaks corresponding to large chromatin fragments comprising thousands of bp. Surprisingly, when we ranked the samples by their H3.1-nucleosome level, we found that those samples with levels below approximately 350 ng/ml contained predominantly mononucleosomes and oligonucleosomes and little or no large chromatin fragments whereas samples with levels above approximately 350 ng/ml contained peaks corresponding to both mononucleosomes and oligonucleosomes as well as large chromatin fragments. These findings are summarised in Table 1 below.

TABLE 1
The correlation of chromatin fragment size distribution with
measured H3.1-nucleosome level in samples obtained
from patients diagnosed with a solid cancer.

| H3.1-nucleosomes ng/ml | Mono-nucleosome peak | Large chromatin peak |
|---|---|---|
| Elevated but <350 ng/ml | | |
| 101 | ++ | − |
| 122 | +++ | − |
| 154 | ++ | − |
| 267 | ++ | + |
| Elevated and >350 ng/ml | | |
| 350 | ++ | − |
| 574 | ++ | ++ |
| 642 | ++ | ++ |
| 1455 | +++ | ++ |

Electropherograms for solid cancer samples with H3.1-nucleosome levels ≤350 ng/ml (shown in FIG. 3A) comprised a strong mono-nucleosome peak (++) and no large chromatin peak or a small narrow peak (+). In contrast, electropherograms for sepsis samples with H3.1-nucleosome levels in the range 101-350 ng/ml (FIG. 5) contained a broad and strong peak corresponding to large chromatin (+++).

Electropherograms for solid cancer samples with H3.1-nucleosome levels >350 ng/ml (shown in FIG. 3B) comprised both a strong mono-nucleosome peak (++) and also a peak corresponding to large chromatin (++).

However, the fragment size profiles for solid cancer samples with H3.1-nucleosome levels >350 ng/ml (shown in FIG. 3B) were not similar to those of the reference NETosis profiles (FIG. 2). Furthermore, the large chromatin peak was smaller and narrower than the corresponding peaks present in electropherograms obtained for sepsis samples with H3.1-nucleosome levels >350 ng/ml shown in FIG. 6 which contained a dominating, broad and strong peak corresponding to large chromatin (++++).

Therefore, a nucleosome/chromatin fragment/cfDNA level measurement in combination with the fragment size distribution profile can be used to distinguish samples taken from solid cancer patients and patients with a NETosis related condition including sepsis.

### Example 4

We similarly investigated EDTA plasma samples obtained from 23 patients diagnosed with sepsis. We measured the H3.1-nucleosome level in the samples by immunoassay and classified them into groups with normal or slightly elevated cf-nucleosome levels (<100 ng/ml), moderately elevated nucleosome levels (100-350 ng/ml) or highly elevated nucleosome levels (>350 ng/ml). DNA was extracted from the plasma samples using a commercial DNA extraction kit and the cfDNA fragment size profile was determined by electrophoresis using the Bioanalyzer system available from Agilent Technologies following the manufacturer's instructions. The results are shown in FIGS. 4-6.

Some of these patients had mild disease with low levels of cf-nucleosomes and others had very high levels. In the electropherograms of all these samples, a large chromatin fragment peak was observed and this peak was the widest, largest and dominant peak present in the electropherograms of the samples.

Surprisingly, the general distribution pattern of circulating chromatin fragment size, particularly relating to large chromatin fragments, was largely independent of the quantity of cf-nucleosomes present, especially for elevated levels of cf-nucleosomes above normal levels as shown in FIGS. 4, 5 and 6 and Table 2. Moreover, the general pattern of the presence of a significant level of large chromatin fragments was true even for samples with low H3.1-nucleosome levels within the normal range as shown in the electropherograms in FIG. 4A for samples containing 29 or 30 ng/ml H3.1-nucleosomes which are remarkably similar in shape to those in FIGS. 4B, 5 and 6 for nucleosome levels up to 734 ng/ml. Moreover, the chromatin fragment profiles observed for the sepsis samples were similar to the NETosis profiles we generated ex vivo shown in FIG. 2. We therefore conclude that a strong, wide, dominant electrophoresis peak at approximately 800 bp or at 1000 bp or higher is indicative of a NETosis related disease in the subject from whom the sample was obtained and that this, in combination with the level of nucleosomes present, can be used to distinguish between an elevated level of nucleosomes consequent to a NETosis related condition and a cancer condition.

TABLE 2
The correlation of chromatin fragment size distribution with
measured H3.1-nucleosome level in samples obtained
from patients diagnosed with sepsis.

| H3.1-nucleosomes ng/ml | Mono-nucleosome peak | Large chromatin peak |
|---|---|---|
| Not significantly elevated | | |
| 29 | ++ | ++ |
| 30 | + | ++ |
| 44 | ++ | ++ |
| 56 | ++ | ++ |
| 56 | + | ++ |
| 59 | ++ | ++ |
| 59 | ++ | +++ |
| 61 | ++ | ++ |
| 66 | ++ | + |
| Elevated but <350 ng/ml | | |
| 72 | ++ | +++ |
| 100 | + | ++++ |
| 194 | ++ | +++ |
| 200 | ++ | ++++ |
| 205 | ++ | +++ |
| 207 | ++ | ++++ |
| 212 | ++ | ++ |
| 229 | ++ | ++ |
| 256 | ++ | +++ |
| 334 | ++ | +++ |
| Elevated and >350 ng/ml | | |
| 398 | ++ | ++++ |
| 577 | ++ | ++++ |
| 641 | + | ++++ |
| 734 | + | ++++ |

### Example 5

We next investigated the circulating chromatin fragment size profile of a haematological cancer patient for comparison with those of patients with sepsis or solid cancers. We measured the cf-nucleosome levels present in EDTA plasma samples collected from 3 subjects diagnosed with metastatic stage IV prostate cancer (PCa), non-Hodgkin's lymphoma (NHL) and sepsis. DNA was extracted from the plasma samples using a commercial DNA extraction kit. The cfDNA fragment size profile was determined by electrophoresis using the Bioanalyzer system available from Agilent Technologies following the manufacturer's instructions.

The electrophoresis results (FIG. 7) showed that the circulating chromatin fragments present in the sepsis plasma sample comprised a dominant peak of large chromatin fragments indicating a NETosis origin for those fragments. In contrast, the PCa sample and the NHL sample contained a strong mononucleosome peak (at approximately 160 bp) and a smaller dinucleosome peak (at approximately 300 bp) but neither contained a significant component of large chromatin fragments. This indicates a cancer associated origin for the chromatin fragments in the PCa and NHL samples. The NHL sample appeared to contain an extremely low proportion of large chromatin fragments despite the high level of H3.1-nucleosomes present in the sample (744 ng/ml). This is different to the pattern we observed for solid cancers and surprising given the reported inflammatory aetiology of haematological cancer disease.

These findings indicate that a high level of H3.1-nucleosomes in a sample obtained from a subject in combination with a fragment profile consisting largely of mononucleosomes with no significant large chromatin component is indicative of a cancer disease in the subject and further indicative that the cancer is a haematological cancer. These findings also illustrate the strong sensitivity and particular utility of the current invention for the differential diagnosis of a subject with an elevated level of circulating chromatin fragments that may be suspected to have an inflammatory or a haematological cancer disease.

### Example 6

In order to confirm the results of EXAMPLE 5 and FIG. 7 for a single NHL sample, we next investigated the circulating chromatin fragment size profile of EDTA plasma samples with high and low H3.1-nucleosome levels obtained from 7 further patients diagnosed with NHL and 5 healthy donors as described in EXAMPLE 5 above. The level of circulating nucleosomes observed in all the NHL patients investigated was elevated (≥100 ng/ml) above that observed in healthy patients (<84 ng/ml) (Table 3). The electropherograms produced for the DNA fragment size profiles are shown in FIG. 8. The profiles were similar to the single result observed for NHL in EXAMPLE 5.

We observed that DNA fragment size profiles for plasma samples obtained from NHL patients contained peaks corresponding only or predominantly to mononucleosomes and oligonucleosomes with no peak, or very small peaks, corresponding to large chromatin fragments. The size of the mononucleosome peaks observed correlated well with the measured H3.1-nucleosome levels (Table 3 and FIG. 8). Moreover, H3.1 nucleosome levels were elevated in all the NHL patients investigated. Therefore, the combination of the circulating nucleosome level together with the DNA fragment size profile is informative of the disease status of the subject. The elevated H3.1-nucleosome level is indicative of a cancer or inflammatory condition in the patient. The lack of any large chromatin peak in any of the samples indicates a probable cancer in each case.

TABLE 3
The correlation of chromatin fragment size distribution with measured
H3.1-nucleosome level in samples obtained from healthy donors and
patients diagnosed with NHL.

| | | H3.1- | Mono- | Large |
|---|---|---|---|---|
| | | nucleosome | nucleosome | chromatin |
| Subject | Disease | level (ng/ml) | peak | peak |
| NHL 1 | Follicular lymphoma | 914 | ++ | − |
| | type 1-2 | | | |
| NHL 2 | DLBCL | 100 | + | − |
| NHL 3 | Follicular lymphoma | 817 | ++ | − |
| | type 3A | | | |
| NHL 4 | DLBCL | 119 | + | − |
| NHL 5 | Follicular lymphoma | 1360 | ++ | − |
| | type 3A | | | |
| NHL 6 | DLBCL (non GCB- | 1450 | ++ | − |
| | type) | | | |
| NHL 7 | Follicular lymphoma, | 270 | + | − |
| | type 3A | | | |
| Healthy | − | 24.5 | + | − |
| donor 1 | | | | |
| Healthy | − | 83.7 | + | − |
| donor 2 | | | | |
| Healthy | − | 74.6 | + | − |
| donor 3 | | | | |
| Healthy | − | 24.3 | + | − |
| donor 4 | | | | |
| Healthy | − | 73.9 | + | − |
| donor 5 | | | | |

DLBCL: Diffuse large B cell lymphoma.

### Example 7

Intercalating dyes bind to free DNA by intercalation in the DNA double helix structure. Protein binding of DNA interferes in binding of intercalating dyes. Polynucleosomes are chains of mononucleosomes linked together by chains of unbound “free” linker DNA and contain more linker DNA, and longer lengths of linker DNA, than mononucleosomes.

We investigated whether DNA intercalating dyes may selectively bind more strongly to polynucleosomes than nucleosomes.

A fluorescent intercalating dye (SYTOX™ Green) was added to recombinant mononucleosomes containing 147, 167 or 187 bp of DNA, HeLa cell derived mononucleosomes or HeLa cell derived polynucleosomes in individual wells of a 96 well plate. 100 μl of 1000 ng/mL of recombinant and mononucleosomes or 500 ng/mL of polynucleosomes in PBS were added to each well in triplicate, followed by the addition of 100 μL of 20 μM SYTOX™ Green in PBS to a final concentration of 500 ng/mL of recombinant and mononucleosomes or 250 ng/mL of polynucleosomes in 10 μM of SYTOX™ Green in 200 μL in each well. Nucleosomes were either kept at room temperature or heated at 95° C. for 30 seconds. Nucleosomes and SYTOX™ green were incubated for 15 minutes and fluorescence was measured following excitation at 510 nm with fluorescent emission measured at 550 nm using a fluorescent micro-titre plate reader.

The results are shown in FIG. 9. A large fluorescent signal was generated by SYTOX™ Green binding of 250ng of polynucleosomes. The signal generated by binding to 500ng of recombinant mononucleosomes was lower. There were no significant differences in fluorescent signal generated from intercalation between recombinant nucleosomes containing 147, 167 or 187 bp of DNA. The signal generated by maximally incorporated dye in 500ng of phage DNA (free DNA) is also shown. The results were unaffected by a 30 second heat treatment.

### Example 8

An antibody directed to bind to a nucleosome, or an epigenetic feature of a nucleosome including a histone isoform or a post-translational histone modification, is coated onto a solid support such as a micro-titre well or a magnetic bead by standard methods in the art. Plasma samples containing elevated levels of nucleosomes of unknown origin are added to wells or beads and incubated at room temperature for 1 hour. The sample is then removed by decanting from the wells, or by magnetically isolating the magnetic beads. Optionally, the solid phase is washed. A DNA intercalating dye, such as SYTOX™ Green, is added to the solid phase and incubated for 15 minutes. The solid phase is washed (again) and the nucleosome bound SYTOX™ green is measured using a fluorimeter or fluorescent micro-titre plate-reader with excitation at 510 nm and fluorescent emission measured at 550 nm.

Samples with elevated nucleosome levels and high fluorescent readings are classified as containing high levels of large polynucleosomes chromatin fragments. The nucleosomes from these subjects are derived predominantly from inflammatory processes, e.g. through NETosis and the production of NETs or ETs.

Samples with elevated nucleosome levels and low fluorescent readings are classified as containing high levels of mononucleosomes. The nucleosomes from these subjects are derived predominantly from cancer processes.

Therefore, a DNA intercalating dye may be used in methods of the invention to determine a cancer or inflammatory origin of chromatin fragments in a patient sample.

1. A method, comprising: determining a size profile of circulating chromatin fragments, cell free nucleosomes (cf-nucleosomes) or cell free DNA (cfDNA) present in a blood, serum or plasma sample, and using the size profile for differential diagnosis of cancer or a NETosis related disease.

2. The method of claim 1, wherein said using comprises using the size profile in combination with a level of circulating chromatin fragments.

3. The method of claim 1, wherein the cancer is a haematological cancer.

4. The method of claim 1, wherein the NETosis related disease is selected from the group consisting of sepsis, COVID-19, influenza, SIRS, ARDS, SARS and pneumonia.

5. The method of claim 1, wherein said determining comprises obtaining the size profile by an electrophoresis method, a DNA sequencing method or using a DNA intercalating dye.

6. (canceled)

7. The method of claim 1, further comprising measuring or detecting the level of circulating cell free nucleosomes in the blood, serum or plasma sample prior to determining the size profile.

8. The method of claim 7, wherein said measuring or detecting is via a method selected from an immunoassay, immunochemical, mass spectroscopy, chromatographic, chromatin immunoprecipitation and a biosensor.

9. The method of claim 7, wherein the measuring or detecting employs a single binding agent or a 2-site immunometric assay employing two binding agents.

10. (canceled)

11. The method of claim 9, wherein the binding agent is selected from the group consisting of a chromatin protein, an antibody, and an agent that binds a histone, a histone isoform, nucleosome core protein, DNA epitope or a protein adducted to a nucleosome.

12-14. (canceled)

15. The method of claim 1, wherein the histone isoform is H3.1.

16. The method of claim 7, wherein the size profile of the circulating chromatin fragments is used in combination with a level of circulating chromatin fragments to indicate whether the disease present is cancer or a NETosis related disease.

17. The method of claim 7, wherein the method further comprises extracting cfDNA from the circulating chromatin fragments in the sample and sequencing the extracted cfDNA.

18. The method of claim 17, wherein said sequencing comprises Next Generation Sequencing (NGS).

19. The method of claim 17, wherein the size profile of the circulating chromatin fragments is used in combination with analysis of the sequenced cfDNA to indicate whether the disease present is cancer or a NETosis related disease.

20-21. (canceled)

22. A method of treating a cancer in a subject, which comprises the following steps:
(i) determining a size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is a cancer; wherein the size profile of the circulating chromatin fragments in a subject with a cancer is smaller compared to a subject with a NETosis related disease; and
(iii) administering a treatment to the subject if based on step (ii) cancer is present.

23. A method of treating a NETosis related disease in a subject, which comprises the following steps:
(i) determining a size profile of circulating chromatin fragments in a blood, serum or plasma sample obtained from the subject, wherein the subject has been determined to have an elevated level of circulating chromatin fragments;
(ii) using the size profile of the circulating chromatin fragments to indicate whether the disease present is a NETosis related disease; wherein the size profile of the circulating chromatin fragments in a subject with a NETosis related disease is larger compared to a subject with cancer; and
(iii) administering a treatment to the subject if based on step (ii) a NETosis related disease is present.

24. (canceled)

25. A method for measuring nucleosomes in a sample, comprising:
(i) contacting the sample with a DNA intercalating dye;
(ii) contacting the sample with a binding agent which specifically binds to a nucleosome or a component thereof;
(iii) determining a degree of binding of the DNA intercalating dye to the nucleosomes; and
(iv) using the degree of binding of the DNA intercalating dye to measure an amount of nucleosomes present in the sample.

26. (canceled)

27. The method of claim 25, wherein the amount of nucleosomes is measured by mass spectrometry or DNA sequencing.

28-29. (canceled)

30. The method of any one of claims 24 to 29, wherein the binding agent is an antibody, a chromatin protein, an agent that binds a histone, a histone isoform, nucleosome core protein, DNA epitope or a protein adducted to a nucleosome.

31-34. (canceled)

35. The method of claim 25, wherein step (ii) is performed before step (i).
